SEQUENCE LISTING (192 sequences)

SEQ ID NO: 1 - protein, 8 aa, synthetic construct
  source 1..8: note = Description of Artificial Sequence: Synthetic peptide
FSQKQQPV

SEQ ID NO: 2 - protein, 8 aa, synthetic construct
  source 1..8: note = Description of Artificial Sequence: Synthetic peptide
FLQQQCSP

SEQ ID NO: 3 - protein, 8 aa, synthetic construct
  source 1..8: note = Description of Artificial Sequence: Synthetic peptide
FSQQQQLV

SEQ ID NO: 4 - protein, 8 aa, synthetic construct
  source 1..8: note = Description of Artificial Sequence: Synthetic peptide
FSQQQQPV

SEQ ID NO: 5 - protein, 8 aa, synthetic construct
  source 1..8: note = Description of Artificial Sequence: Synthetic peptide
FPQQQQPI

SEQ ID NO: 6 - protein, 8 aa, synthetic construct
  source 1..8: note = Description of Artificial Sequence: Synthetic peptide
FSQQQQPP

SEQ ID NO: 7 - protein, 8 aa, synthetic construct
  source 1..8: note = Description of Artificial Sequence: Synthetic peptide
FSQQQQPS

SEQ ID NO: 8 - protein, 8 aa, synthetic construct
  source 1..8: note = Description of Artificial Sequence: Synthetic peptide
FPQQQIPV

SEQ ID NO: 9 - protein, 8 aa, synthetic construct
  source 1..8: note = Description of Artificial Sequence: Synthetic peptide
FSQQQQSP

SEQ ID NO: 10 - skipped

SEQ ID NO: 11 - protein, 8 aa, synthetic construct
  source 1..8: note = Description of Artificial Sequence: Synthetic peptide
FSRQEKSP

SEQ ID NO: 12 - protein, 8 aa, synthetic construct
  source 1..8: note = Description of Artificial Sequence: Synthetic peptide
SSEQEHSP

SEQ ID NO: 13 - protein, 8 aa, synthetic construct
  source 1..8: note = Description of Artificial Sequence: Synthetic peptide
FSHQRPPF

SEQ ID NO: 14 - protein, 8 aa, synthetic construct
  source 1..8: note = Description of Artificial Sequence: Synthetic peptide
FSKQEQQQ

SEQ ID NO: 15 - protein, 8 aa, synthetic construct
  source 1..8: note = Description of Artificial Sequence: Synthetic peptide
FSKQTQPV

SEQ ID NO: 16 - protein, 8 aa, synthetic construct
  source 1..8: note = Description of Artificial Sequence: Synthetic peptide
FSRQHQPS

SEQ ID NO: 17 - protein, 8 aa, synthetic construct
  source 1..8: note = Description of Artificial Sequence: Synthetic peptide
FSRQQKSP

SEQ ID NO: 18 - protein, 8 aa, synthetic construct
  source 1..8: note = Description of Artificial Sequence: Synthetic peptide
SSEQQHSP

SEQ ID NO: 19 - protein, 8 aa, synthetic construct
  source 1..8: note = Description of Artificial Sequence: Synthetic peptide
FSHQQRSG

SEQ ID NO: 20 - protein, 8 aa, synthetic construct
  source 1..8: note = Description of Artificial Sequence: Synthetic peptide
DSHQQHSP

SEQ ID NO: 21 - protein, 9 aa, synthetic construct
  source 1..9: note = Description of Artificial Sequence: Synthetic peptide
LFSQKQQPV

SEQ ID NO: 22 - protein, 9 aa, synthetic construct
  source 1..9: note = Description of Artificial Sequence: Synthetic peptide
QFPQQQIPV

SEQ ID NO: 23 - protein, 9 aa, synthetic construct
  source 1..9: note = Description of Artificial Sequence: Synthetic peptide
RFPQQQIPV

SEQ ID NO: 24 - protein, 9 aa, synthetic construct
  source 1..9: note = Description of Artificial Sequence: Synthetic peptide
PFLQQQQPA

SEQ ID NO: 25 - protein, 9 aa, synthetic construct
  source 1..9: note = Description of Artificial Sequence: Synthetic peptide
PFLQQQQPP

SEQ ID NO: 26 - protein, 9 aa, synthetic construct
  source 1..9: note = Description of Artificial Sequence: Synthetic peptide
PFLQQQQPS

SEQ ID NO: 27 - protein, 9 aa, synthetic construct
  source 1..9: note = Description of Artificial Sequence: Synthetic peptide
PFLQQQQPV

SEQ ID NO: 28 - protein, 9 aa, synthetic construct
  source 1..9: note = Description of Artificial Sequence: Synthetic peptide
PFPQQQQPI

SEQ ID NO: 29 - protein, 9 aa, synthetic construct
  source 1..9: note = Description of Artificial Sequence: Synthetic peptide
PFPQQQQPL

SEQ ID NO: 30 - protein, 9 aa, synthetic construct
  source 1..9: note = Description of Artificial Sequence: Synthetic peptide
PFPQQQQPP

SEQ ID NO: 31 - protein, 9 aa, synthetic construct
  source 1..9: note = Description of Artificial Sequence: Synthetic peptide
PFPQQQQPV

SEQ ID NO: 32 - protein, 9 aa, synthetic construct
  source 1..9: note = Description of Artificial Sequence: Synthetic peptide
PFSQHQQPV

SEQ ID NO: 33 - protein, 9 aa, synthetic construct
  source 1..9: note = Description of Artificial Sequence: Synthetic peptide
PFSQQQHPV

SEQ ID NO: 34 - protein, 9 aa, synthetic construct
  source 1..9: note = Description of Artificial Sequence: Synthetic peptide
PFSQQQQPA

SEQ ID NO: 35 - protein, 9 aa, synthetic construct
  source 1..9: note = Description of Artificial Sequence: Synthetic peptide
PFSQQQQPF

SEQ ID NO: 36 - protein, 9 aa, synthetic construct
  source 1..9: note = Description of Artificial Sequence: Synthetic peptide
PFSQQQQPI

SEQ ID NO: 37 - protein, 9 aa, synthetic construct
  source 1..9: note = Description of Artificial Sequence: Synthetic peptide
PFSQQQQPL

SEQ ID NO: 38 - protein, 9 aa, synthetic construct
  source 1..9: note = Description of Artificial Sequence: Synthetic peptide
PFSQQQQPP

SEQ ID NO: 39 - protein, 9 aa, synthetic construct
  source 1..9: note = Description of Artificial Sequence: Synthetic peptide
PFSQQQQPQ

SEQ ID NO: 40 - protein, 9 aa, synthetic construct
  source 1..9: note = Description of Artificial Sequence: Synthetic peptide
PFSQQQQPS

SEQ ID NO: 41 - protein, 9 aa, synthetic construct
  source 1..9: note = Description of Artificial Sequence: Synthetic peptide
PFSQQQQPV

SEQ ID NO: 42 - protein, 9 aa, synthetic construct
  source 1..9: note = Description of Artificial Sequence: Synthetic peptide
PFSQQQSPF

SEQ ID NO: 43 - protein, 9 aa, synthetic construct
  source 1..9: note = Description of Artificial Sequence: Synthetic peptide
PFSQQRQPV

SEQ ID NO: 44 - protein, 9 aa, synthetic construct
  source 1..9: note = Description of Artificial Sequence: Synthetic peptide
PFSQQTQPV

SEQ ID NO: 45 - protein, 9 aa, synthetic construct
  source 1..9: note = Description of Artificial Sequence: Synthetic peptide
PISQQQQPP

SEQ ID NO: 46 - protein, 9 aa, synthetic construct
  source 1..9: note = Description of Artificial Sequence: Synthetic peptide
PISQQQQPQ

SEQ ID NO: 47 - protein, 9 aa, synthetic construct
  source 1..9: note = Description of Artificial Sequence: Synthetic peptide
PLSQQQQPP

SEQ ID NO: 48 - protein, 9 aa, synthetic construct
  source 1..9: note = Description of Artificial Sequence: Synthetic peptide
PLSQQQQPV

SEQ ID NO: 49 - skipped

SEQ ID NO: 50 - protein, 9 aa, synthetic construct
  source 1..9: note = Description of Artificial Sequence: Synthetic peptide
FSQQQLPPF

SEQ ID NO: 51 - protein, 9 aa, synthetic construct
  source 1..9: note = Description of Artificial Sequence: Synthetic peptide
FSQQQQIPV

SEQ ID NO: 52 - protein, 9 aa, synthetic construct
  source 1..9: note = Description of Artificial Sequence: Synthetic peptide
FSQQQQPPF

SEQ ID NO: 53 - protein, 9 aa, synthetic construct
  source 1..9: note = Description of Artificial Sequence: Synthetic peptide
FSQQQQSPF

SEQ ID NO: 54 - protein, 9 aa, synthetic construct
  source 1..9: note = Description of Artificial Sequence: Synthetic peptide
FSQQQQTPF

SEQ ID NO: 55 - protein, 9 aa, synthetic construct
  source 1..9: note = Description of Artificial Sequence: Synthetic peptide
ISQQQQPPL

SEQ ID NO: 56 - protein, 9 aa, synthetic construct
  source 1..9: note = Description of Artificial Sequence: Synthetic peptide
FSQQQQQPF

SEQ ID NO: 57 - protein, 9 aa, synthetic construct
  source 1..9: note = Description of Artificial Sequence: Synthetic peptide
FSQQQQQPI

SEQ ID NO: 58 - protein, 9 aa, synthetic construct
  source 1..9: note = Description of Artificial Sequence: Synthetic peptide
FSQQQQQPL

SEQ ID NO: 59 - protein, 9 aa, synthetic construct
  source 1..9: note = Description of Artificial Sequence: Synthetic peptide
FSQQQQQPP

SEQ ID NO: 60 - protein, 9 aa, synthetic construct
  source 1..9: note = Description of Artificial Sequence: Synthetic peptide
FSQQQQQPV

SEQ ID NO: 61 - protein, 9 aa, synthetic construct
  source 1..9: note = Description of Artificial Sequence: Synthetic peptide
FSRQQQIPV

SEQ ID NO: 62 - protein, 9 aa, synthetic construct
  source 1..9: note = Description of Artificial Sequence: Synthetic peptide
FLQQQCSPM

SEQ ID NO: 63 - protein, 9 aa, synthetic construct
  source 1..9: note = Description of Artificial Sequence: Synthetic peptide
FLQQQCSPV

SEQ ID NO: 64 - protein, 9 aa, synthetic construct
  source 1..9: note = Description of Artificial Sequence: Synthetic peptide
FSQQQQLVL

SEQ ID NO: 65 - skipped

SEQ ID NO: 66 - protein, 15 aa, synthetic construct
  source 1..15: note = Description of Artificial Sequence: Synthetic peptide
QQPPFSQQQQPVLPQ

SEQ ID NO: 67 - protein, 15 aa, synthetic construct
  source 1..15: note = Description of Artificial Sequence: Synthetic peptide
QQPPFSEQEQPVLPQ

SEQ ID NO: 68 - protein, 15 aa, synthetic construct
  source 1..15: note = Description of Artificial Sequence: Synthetic peptide
QPPFSQQQQSPFSQQ

SEQ ID NO: 69 - protein, 15 aa, synthetic construct
  source 1..15: note = Description of Artificial Sequence: Synthetic peptide
QPPFSEQQESPFSQQ

SEQ ID NO: 70 - protein, 15 aa, synthetic construct
  source 1..15: note = Description of Artificial Sequence: Synthetic peptide
QQPPFSHQRPPFSQQ

SEQ ID NO: 71 - protein, 15 aa, synthetic construct
  source 1..15: note = Description of Artificial Sequence: Synthetic peptide
QQPPFSKQEQQQPIL

SEQ ID NO: 72 - protein, 15 aa, synthetic construct
  source 1..15: note = Description of Artificial Sequence: Synthetic peptide
QGPPFSKQTQPVIPQ

SEQ ID NO: 73 - protein, 15 aa, synthetic construct
  source 1..15: note = Description of Artificial Sequence: Synthetic peptide
QQPAFSRQHQPSLPQ

SEQ ID NO: 74 - protein, 15 aa, synthetic construct
  source 1..15: note = Description of Artificial Sequence: Synthetic peptide
QQPPFPRQHQQFPQQ

SEQ ID NO: 75 - protein, 15 aa, synthetic construct
  source 1..15: note = Description of Artificial Sequence: Synthetic peptide
QQPFPQEQRSQFSQQ

SEQ ID NO: 76 - protein, 15 aa, synthetic construct
  source 1..15: note = Description of Artificial Sequence: Synthetic peptide
QQPPFWQHQPPFSQQ

SEQ ID NO: 77 - protein, 15 aa, synthetic construct
  source 1..15: note = Description of Artificial Sequence: Synthetic peptide
QQPPFSEQEQPSSQQ

SEQ ID NO: 78 - protein, 15 aa, synthetic construct
  source 1..15: note = Description of Artificial Sequence: Synthetic peptide
QQSPFSRQEKSPFSQ

SEQ ID NO: 79 - protein, 15 aa, synthetic construct
  source 1..15: note = Description of Artificial Sequence: Synthetic peptide
QQPPSSEQEHSPPSQ

SEQ ID NO: 80 - protein, 15 aa, synthetic construct
  source 1..15: note = Description of Artificial Sequence: Synthetic peptide
QSPFSRQQKSPFSQQ

SEQ ID NO: 81 - protein, 15 aa, synthetic construct
  source 1..15: note = Description of Artificial Sequence: Synthetic peptide
QPPSSEQQHSPPSQQ

SEQ ID NO: 82 - protein, 15 aa, synthetic construct
  source 1..15: note = Description of Artificial Sequence: Synthetic peptide
QPPFSHQRPPFSQQQ

SEQ ID NO: 83 - protein, 15 aa, synthetic construct
  source 1..15: note = Description of Artificial Sequence: Synthetic peptide
QPFFSHQQRSGFSQQ

SEQ ID NO: 84 - protein, 15 aa, synthetic construct
  source 1..15: note = Description of Artificial Sequence: Synthetic peptide
QPPDSHQQHSPGSQQ

SEQ ID NO: 85 - protein, 15 aa, synthetic construct
  source 1..15: note = Description of Artificial Sequence: Synthetic peptide
QQPFPEQQRSQFSQQ

SEQ ID NO: 86 - protein, 15 aa, synthetic construct
  source 1..15: note = Description of Artificial Sequence: Synthetic peptide
QPSFSRQQKQPQSQQ

SEQ ID NO: 87 - protein, 15 aa, synthetic construct
  source 1..15: note = Description of Artificial Sequence: Synthetic peptide
QPPFSEQQEPSSQQP

SEQ ID NO: 88 - protein, 281 aa, Triticum aestivum
QMETSCIPGLERPWQQQPLQQKETFPQQPPSSQQQQPFPQQPPFLQQQPSFSQQPLFSQKQQPVLPQQPAFSQQQQTVLPQQPAFPQQQHQQLLQQQIPVVHPSILQQLNPCKVFLQQQCSPVAMPQHLARSQMWQQSSCNVMQQQCCQQLPRIPEQSRYEAIRAIIFSIILQEQQQGFVQPQQQQPQQSVQGVYQPQQQSQQQLGQCSFQQPQQQLGQQPQQQQVQKGIFLQPHQIARLEVMTSIALRTLPTMCSVNVPLYSSITSAPLGVGTGVGAYKG

SEQ ID NO: 89 - protein, 294 aa, Triticum aestivum
QMETRCIPGLERPWQQQPLPPQQTFPQQPLFSQQQQQQLFPQQPSFSQQQPPFWQQQPPFSQQQPILPQQPPFSQQQQLVLPQQPPFSQQQQPVLPPQQSPFPQQQQQHQQLVQQQIPVVQPSILQQLNPCKVFLQQQCSPVAMPQRLARSQMLQQSSCHVMQQQCCQQLPQIPQQSRYEAIRAIIYSIILQEQQQVQGSIQSQQQQPQQLGQCVSQPQQQSQQQLGQQPQQQQLAQGTFLQPHQIAQLEVMTSIALRILPTMCSVNVPLYRTTTSVPFGVGTGSWCLLIRKDL

SEQ ID NO: 90 - protein, 332 aa, Triticum aestivum
QMETSRVPGLEKPWQQQPLPPQQQPPCSQQQQPFPQQQQPIIILQQSPFSQQQQPVLPQQQPVIILQQPPFSQQQQPVLPQQPPFSQQQQQQQQQQPPFSQQQQPVLPQQPPFSQQQQPPFSQQQQPSSQQPPFPQQHQQFPQQQIPVVQPSVLQQLNPCKVFLQQQCSHVAMSQRLARSQMWQQSSCHVMQQQCCQQLPQIPEQSRSEAIRAIVYSIILQEQQQGFVQPQQQQPQQSGQGVSQHQQQSQQQQQLGQCSFQQPQQLQQLRQQPQQQQIPQGIFLQPHQISQLEVMTSIALRTLPTMCGVNVPLYSSTTIMPFSIGTGVGAYK

SEQ ID NO: 91 - protein, 294 aa, synthetic construct
  source 1..294: note = Description of Artificial Sequence: Synthetic polypeptide
QMETRCIPGLERPWQQQPLPPQQTFPQQPLFSQQQQQQLFPQQPSFSQQQPPFWQQQPPFSQQQPILPQQPPDSHQQHSPGPQQPPDSHQQHSPGPPQQSPFPQQQQQHQQLVQQQIPVVQPSILQQLNPCKVDSHQQCSPGAMPQRLARSQMLQQSSCHVMQQQCCQQLPQIPQQSRYEAIRAIIYSIILQEQQQVQGSIQSQQQQPQQLGQCVSQPQQQSQQQLGQQPQQQQLAQGTFLQPHQIAQLEVMTSIALRILPTMCSVNVPLYRTTTSVPFGVGTGSWCLLIRKDL

SEQ ID NO: 92 - protein, 294 aa, synthetic construct
  source 1..294: note = Description of Artificial Sequence: Synthetic polypeptide
QMETRCIPGLERPWQQQPLPPQQTFPQQPLFSQQQQQQLFPQQPSFSQQQPPFWQQQPPFSQQQPILPQQPFFSHQQRSGFPQQPFFSHQQRSGFPPQQSPFPQQQQQHQQLVQQQIPVVQPSILQQLNPCKFFSHQQCSGFAMPQRLARSQMLQQSSCHVMQQQCCQQLPQIPQQSRYEAIRAIIYSIILQEQQQVQGSIQSQQQQPQQLGQCVSQPQQQSQQQLGQQPQQQQLAQGTFLQPHQIAQLEVMTSIALRILPTMCSVNVPLYRTTTSVPFGVGTGSWCLLIRKDL

SEQ ID NO: 93 - protein, 294 aa, synthetic construct
  source 1..294: note = Description of Artificial Sequence: Synthetic polypeptide
QMETRCIPGLERPWQQQPLPPQQTFPQQPLFSQQQQQQLFPQQPSFSQQQPPFWQQQPPFSQQQPILPQQPPFSHQRPPFSQQQPPFSHQRPPFSQPQQSPFPQQQQQHQQLVQQQIPVVQPSILQQLNPCKVFSHQRCPFSQMPQRLARSQMLQQSSCHVMQQQCCQQLPQIPQQSRYEAIRAIIYSIILQEQQQVQGSIQSQQQQPQQLGQCVSQPQQQSQQQLGQQPQQQQLAQGTFLQPHQIAQLEVMTSIALRILPTMCSVNVPLYRTTTSVPFGVGTGSWCLLIRKDL

SEQ ID NO: 94 - protein, 294 aa, synthetic construct
  source 1..294: note = Description of Artificial Sequence: Synthetic polypeptide
QMETRCIPGLERPWQQQPLPPQQTFPQQPLFSQQQQQQLFPQQPSFSQQQPPFWQQQPPFSQQQPILPQGPPFSKQTQPVIPQGPPFSKQTQPVIPPQQSPFPQQQQQHQQLVQQQIPVVQPSILQQLNPGPPFSKQTCPVIAMPQRLARSQMLQQSSCHVMQQQCCQQLPQIPQQSRYEAIRAIIYSIILQEQQQVQGSIQSQQQQPQQLGQCVSQPQQQSQQQLGQQPQQQQLAQGTFLQPHQIAQLEVMTSIALRILPTMCSVNVPLYRTTTSVPFGVGTGSWCLLIRKDL

SEQ ID NO: 95 - protein, 294 aa, synthetic construct
  source 1..294: note = Description of Artificial Sequence: Synthetic polypeptide
QMETRCIPGLERPWQQQPLPPQQTFPQQPLFSQQQQQQLFPQQPSFSQQQPPFWQQQPPFSQQQPILPQQPPSSQQQHSPPSQQPPSSQQQHSPPSPQQSPFPQQQQQHQQLVQQQIPVVQPSILQQLNPCKPSSQQQCSPPSMPQRLARSQMLQQSSCHVMQQQCCQQLPQIPQQSRYEAIRAIIYSIILQEQQQVQGSIQSQQQQPQQLGQCVSQPQQQSQQQLGQQPQQQQLAQGTFLQPHQIAQLEVMTSIALRILPTMCSVNVPLYRTTTSVPFGVGTGSWCLLIRKDL

SEQ ID NO: 96 - protein, 294 aa, synthetic construct
  source 1..294: note = Description of Artificial Sequence: Synthetic polypeptide
QMETRCIPGLERPWQQQPLPPQQTFPQQPLFSQQQQQQLFPQQPSFSQQQPPFWQQQPPFSQQQPILPQQQPFPQQQRSQFPQQQPFPQQQRSQFPPQQSPFPQQQQQHQQLVQQQIPVVQPSILQQLNPCQPFPQQQCSQFAMPQRLARSQMLQQSSCHVMQQQCCQQLPQIPQQSRYEAIRAIIYSIILQEQQQVQGSIQSQQQQPQQLGQCVSQPQQQSQQQLGQQPQQQQLAQGTFLQPHQIAQLEVMTSIALRILPTMCSVNVPLYRTTTSVPFGVGTGSWCLLIRKDL

SEQ ID NO: 97 - protein, 294 aa, synthetic construct
  source 1..294: note = Description of Artificial Sequence: Synthetic polypeptide
QMETRCIPGLERPWQQQPLPPQQTFPQQPLFSQQQQQQLFPQQPSFSQQQPPFWQQQPPFSQQQPILPQQPSFSRQQKQPQPQQPSFSRQQKQPQPPQQSPFPQQQQQHQQLVQQQIPVVQPSILQQLNPCKSFSRQQCQPQAMPQRLARSQMLQQSSCHVMQQQCCQQLPQIPQQSRYEAIRAIIYSIILQEQQQVQGSIQSQQQQPQQLGQCVSQPQQQSQQQLGQQPQQQQLAQGTFLQPHQIAQLEVMTSIALRILPTMCSVNVPLYRTTTSVPFGVGTGSWCLLIRKDL

SEQ ID NO: 98 - protein, 294 aa, synthetic construct
  source 1..294: note = Description of Artificial Sequence: Synthetic polypeptide
QMETRCIPGLERPWQQQPLPPQQTFPQQPLFSQQQQQQLFPQQPSFSQQQPPFWQQQPPFSQQQPILPQQSPFSRQQKSPFSQQSPFSRQQKSPFSPQQSPFPQQQQQHQQLVQQQIPVVQPSILQQLNPCSPFSRQQCSPFSMPQRLARSQMLQQSSCHVMQQQCCQQLPQIPQQSRYEAIRAIIYSIILQEQQQVQGSIQSQQQQPQQLGQCVSQPQQQSQQQLGQQPQQQQLAQGTFLQPHQIAQLEVMTSIALRILPTMCSVNVPLYRTTTSVPFGVGTGSWCLLIRKDL

SEQ ID NO: 99 - protein, 332 aa, synthetic construct
  source 1..332: note = Description of Artificial Sequence: Synthetic polypeptide
QMETSRVPGLEKPWQQQPLPPQQQPPCSQQQQAFSRQHQPSIILQQSAFSRQHQPSLPQQQPVIILQQPAFSRQHQPSLPQQPPFSQQQQQQQQQQPAFSRQHQPSLPQQPAFSRQHQPSFSQQQQPSSQQPPFPQQHQAFSRQHQPSVQPSVLQQLNPCKVFLQQQCSHVAMSQRLARSQMWQQSSCHVMQQQCCQQLPQIPEQSRSEAIRAIVYSIILQEQQQGFVQPQQQQPQQSGQGVSQHQQQSQQQQQLGQCSFQQPQQLQQLRQQPQQQQIPQGIFLQPHQISQLEVMTSIALRTLPTMCGVNVPLYSSTTIMPFSIGTGVGAYK

SEQ ID NO: 100 - protein, 281 aa, synthetic construct
  source 1..281: note = Description of Artificial Sequence: Synthetic polypeptide
QMETSCIPGLERPWQQQPLQQKETFPQQPPSSQQQQPFPQQPPFLQQQPSFSQQPAFSRQHQPSLPQQPAFSQQQQTVLPQQPAFPQQQHQQLLQQQIPVVHPSILQQLNPCKAFSRQHCPSVAMPQHLARSQMWQQSSCNVMQQQCCQQLPRIPEQSRYEAIRAIIFSIILQEQQQGFVQPQQQQPQQSVQGVYQPQQQSQQQLGQCSFQQPQQQLGQQPQQQQVQKGIFLQPHQIARLEVMTSIALRTLPTMCSVNVPLYSSITSAPLGVGTGVGAYKG

SEQ ID NO: 101 - protein, 281 aa, synthetic construct
  source 1..281: note = Description of Artificial Sequence: Synthetic polypeptide
QMETSCIPGLERPWQQQPLQQKETFPQQPPSSQQQQPFPQQPPFLQQQPSFSQQPLDSHQQHSPGPQQPAFSQQQQTVLPQQPAFPQQQHQQLLQQQIPVVHPSILQQLNPCKVDSHQQCSPGAMPQHLARSQMWQQSSCNVMQQQCCQQLPRIPEQSRYEAIRAIIFSIILQEQQQGFVQPQQQQPQQSVQGVYQPQQQSQQQLGQCSFQQPQQQLGQQPQQQQVQKGIFLQPHQIARLEVMTSIALRTLPTMCSVNVPLYSSITSAPLGVGTGVGAYKG

SEQ ID NO: 102 - protein, 281 aa, synthetic construct
  source 1..281: note = Description of Artificial Sequence: Synthetic polypeptide
QMETSCIPGLERPWQQQPLQQKETFPQQPPSSQQQQPFPQQPPFLQQQPSFSQQPFFSHQQRSGFPQQPAFSQQQQTVLPQQPAFPQQQHQQLLQQQIPVVHPSILQQLNPCKFFSHQQCSGFAMPQHLARSQMWQQSSCNVMQQQCCQQLPRIPEQSRYEAIRAIIFSIILQEQQQGFVQPQQQQPQQSVQGVYQPQQQSQQQLGQCSFQQPQQQLGQQPQQQQVQKGIFLQPHQIARLEVMTSIALRTLPTMCSVNVPLYSSITSAPLGVGTGVGAYKG

SEQ ID NO: 103 - protein, 281 aa, synthetic construct
  source 1..281: note = Description of Artificial Sequence: Synthetic polypeptide
QMETSCIPGLERPWQQQPLQQKETFPQQPPSSQQQQPFPQQPPFLQQQPSFSQQPLFSHQRPPFSQQQPAFSQQQQTVLPQQPAFPQQQHQQLLQQQIPVVHPSILQQLNPCKVFSHQRCPFSQMPQHLARSQMWQQSSCNVMQQQCCQQLPRIPEQSRYEAIRAIIFSIILQEQQQGFVQPQQQQPQQSVQGVYQPQQQSQQQLGQCSFQQPQQQLGQQPQQQQVQKGIFLQPHQIARLEVMTSIALRTLPTMCSVNVPLYSSITSAPLGVGTGVGAYKG

SEQ ID NO: 104 - protein, 281 aa, synthetic construct
  source 1..281: note = Description of Artificial Sequence: Synthetic polypeptide
QMETSCIPGLERPWQQQPLQQKETFPQQPPSSQQQQPFPQQPPFLQQQPSFSQGPPFSKQTQPVIPQQPAFSQQQQTVLPQQPAFPQQQHQQLLQQQIPVVHPSILQQLNPCPPFSKQTCPVIAMPQHLARSQMWQQSSCNVMQQQCCQQLPRIPEQSRYEAIRAIIFSIILQEQQQGFVQPQQQQPQQSVQGVYQPQQQSQQQLGQCSFQQPQQQLGQQPQQQQVQKGIFLQPHQIARLEVMTSIALRTLPTMCSVNVPLYSSITSAPLGVGTGVGAYKG

SEQ ID NO: 105 - protein, 281 aa, synthetic construct
  source 1..281: note = Description of Artificial Sequence: Synthetic polypeptide
QMETSCIPGLERPWQQQPLQQKETFPQQPPSSQQQQPFPQQPPFLQQQPSFSQQPPSSQQQHSPPSQQPAFSQQQQTVLPQQPAFPQQQHQQLLQQQIPVVHPSILQQLNPCKPSSQQQCSPPSMPQHLARSQMWQQSSCNVMQQQCCQQLPRIPEQSRYEAIRAIIFSIILQEQQQGFVQPQQQQPQQSVQGVYQPQQQSQQQLGQCSFQQPQQQLGQQPQQQQVQKGIFLQPHQIARLEVMTSIALRTLPTMCSVNVPLYSSITSAPLGVGTGVGAYKG

SEQ ID NO: 106 - protein, 281 aa, synthetic construct
  source 1..281: note = Description of Artificial Sequence: Synthetic polypeptide
QMETSCIPGLERPWQQQPLQQKETFPQQPPSSQQQQPFPQQPPFLQQQPSFSQQQPFPQQQRSQFPQQPAFSQQQQTVLPQQPAFPQQQHQQLLQQQIPVVHPSILQQLNPCQPFPQQQCSQFAMPQHLARSQMWQQSSCNVMQQQCCQQLPRIPEQSRYEAIRAIIFSIILQEQQQGFVQPQQQQPQQSVQGVYQPQQQSQQQLGQCSFQQPQQQLGQQPQQQQVQKGIFLQPHQIARLEVMTSIALRTLPTMCSVNVPLYSSITSAPLGVGTGVGAYKG

SEQ ID NO: 107 - protein, 281 aa, synthetic construct
  source 1..281: note = Description of Artificial Sequence: Synthetic polypeptide
QMETSCIPGLERPWQQQPLQQKETFPQQPPSSQQQQPFPQQPPFLQQQPSFSQQPSFSRQQKQPQPQQPAFSQQQQTVLPQQPAFPQQQHQQLLQQQIPVVHPSILQQLNPCKSFSRQQCQPQAMPQHLARSQMWQQSSCNVMQQQCCQQLPRIPEQSRYEAIRAIIFSIILQEQQQGFVQPQQQQPQQSVQGVYQPQQQSQQQLGQCSFQQPQQQLGQQPQQQQVQKGIFLQPHQIARLEVMTSIALRTLPTMCSVNVPLYSSITSAPLGVGTGVGAYKG

SEQ ID NO: 108 - protein, 281 aa, synthetic construct
  source 1..281: note = Description of Artificial Sequence: Synthetic polypeptide
QMETSCIPGLERPWQQQPLQQKETFPQQPPSSQQQQPFPQQPPFLQQQPSFSQQSPFSRQQKSPFSQQPAFSQQQQTVLPQQPAFPQQQHQQLLQQQIPVVHPSILQQLNPCSPFSRQQCSPFSMPQHLARSQMWQQSSCNVMQQQCCQQLPRIPEQSRYEAIRAIIFSIILQEQQQGFVQPQQQQPQQSVQGVYQPQQQSQQQLGQCSFQQPQQQLGQQPQQQQVQKGIFLQPHQIARLEVMTSIALRTLPTMCSVNVPLYSSITSAPLGVGTGVGAYKG

SEQ ID NO: 109 - protein, 294 aa, synthetic construct
  source 1..294: note = Description of Artificial Sequence: Synthetic polypeptide
QMETRCIPGLERPWQQQPLPPQQTFPQQPLFSQQQQQQLFPQQPSFSQQQPPFWQQQPPFSQQQPILPQQPAFSRQHQPSLPQQPAFSRQHQPSLPPQQSPFPQQQQQHQQLVQQQIPVVQPSILQQLNPCKAFSRQHCPSVAMPQRLARSQMLQQSSCHVMQQQCCQQLPQIPQQSRYEAIRAIIYSIILQEQQQVQGSIQSQQQQPQQLGQCVSQPQQQSQQQLGQQPQQQQLAQGTFLQPHQIAQLEVMTSIALRILPTMCSVNVPLYRTTTSVPFGVGTGSWCLLIRKDL

SEQ ID NO: 110 - protein, 281 aa, synthetic construct
  source 1..281: note = Description of Artificial Sequence: Synthetic polypeptide
QMETSCIPGLERPWQQQPLQQKETFPQQPPSSQQQHSPPQQPPFLQQQPSFSQQPFFSRQQKSPFPQQPPFSKQQQQQLPQQPAFPQQQHQQLLQQQIPVVHPSILQQLNPCKVFSRQQKSPFAMPQHLARSQMWQQSSCNVMQQQCCQQLPRIPEQSRYEAIRAIIFSIILQEQQQGFVQPQQQQPQQSVQGVYQPQQQSQQQLGQCSFQQPQQQLGQQPQQQQVQKGIFLQPHQIARLEVMTSIALRTLPTMCSVNVPLYSSITSAPLGVGTGVGAYKG

SEQ ID NO: 111 - protein, 281 aa, synthetic construct
  source 1..281: note = Description of Artificial Sequence: Synthetic polypeptide
QMETSCIPGLERPWQQQPLQQKETFPQQPPSSQQQHSPPQQPPFLQQQPSFSQQPSSSQQQHSPPPQQPPFSKQQQQQLPQQPAFPQQQHQQLLQQQIPVVHPSILQQLNPCKVSSQQQHSPPAMPQHLARSQMWQQSSCNVMQQQCCQQLPRIPEQSRYEAIRAIIFSIILQEQQQGFVQPQQQQPQQSVQGVYQPQQQSQQQLGQCSFQQPQQQLGQQPQQQQVQKGIFLQPHQIARLEVMTSIALRTLPTMCSVNVPLYSSITSAPLGVGTGVGAYKG

SEQ ID NO: 112 - protein, 281 aa, Triticum aestivum
QMDTSCIPGLERPWQQQPLPPQQTFPQQPPFSQQQQQPFPQQPSFSQQQPPFSQQQPILPQGPPFSQQTQPVLPQQSPFSQQQQLTLPPQQQQQLPQQQISIVQPSILQQLSPCKVFLQQQCSPMAMPQRLARSQMWQQSSCHVMQQQCCQQLSQIPEQSRYDAIRAITYSIILQEQQQGFVQAQQQQPQQSGQGVSQSQQQSQQQLGQCSFQQPQQQLGQQPQEQQVQQGTFLQPHQIAHLEVMTSIALRTLPTMCSVNVPLYSSTTSVPFGVGTGVGAY

SEQ ID NO: 113 - protein, 281 aa, Triticum aestivum
QMETSRVPGLEKPWQQQPLPPQQQPPCSQQQQPFPQQQQPIIILQQSPFSQQQQPVLPQQPPFSQQQQPPFSQQQQPSSQQPPFPQQHQQFPQQQIPVVQPSVLQQLNPCKVFLQQQCSHVAMSQRLARSQMWQQSSCHVMQQQCCQQLPQIPEQSRSEAIRAIVYSIILQEQQQGFVQPQQQQPQQSGQGVSQHQQQSQQQQQLGQCSFQQPQQLQQLGQQPQQQQIPQGIFLQPHQISQLEVMTSIALRTLPTMCGVNVPLYSSTTIMPFSIGTGVGAY

SEQ ID NO: 114 - protein, 277 aa, Triticum aestivum
QMETSCIPGLERPWQEQTLPPQQTLFPQQQPFPQQQQQQPPFSQQQPSFSQQQPILPQLPFSQQQQPVLPQQSPFSQQQLVLPPQQQYQQLLQQQIPIVQPSVLQQLNPCKVFLQQQCNPVAMPQHLARSQMWQQSSCHVMQQQCCQQLPQIPEQSRYDAIRAITYSIILQEQQQGFVQAQQQQPQQLGQGVSQSQQQSQQQLGQCSFQQPQQQLGQQPQQQQVLQGTFLQPHQIAHLEVMTSIALRTLPMMCSVNVPLYSSTTSVPFSVGTGVGAY

SEQ ID NO: 115 - protein, 275 aa, Triticum aestivum
QMETSCIPGLERPWQEQTLPPQQTLFPQQQPFPQQQQPPFSQQQPSFLQQQPILPQLPFSQQQQPVLPQQSPFSQQQLVLPPQQQYQQVLQQQIPIVQPSVLQQLNPCKVFLQQQCNPVAMPQRLARSQMWQQSSCHVMQQQCCQQLPQIPEQSRYDAIRAITYSIILQEQQQGFVQAQQQQPQQLGQGVSQSQQQSQQQLGQCSFQQPQQQLGQQPQQQQVLQGTFLQPHQIAHLEVMTSIALRTLPMMCSVNVPLYSSTTSVPFSIGTGVGAY

SEQ ID NO: 116 - protein, 331 aa, Triticum aestivum
QMETSHIPSLEKPLQQQPLPLQQILWYQQQQPIQQQPQPFPQQPPCSQQQQPPLSQQQQPPFSQQQPPFSQQQQPVLPQQPPFSQQQQQFPQQQQPLLPQQPPFSQQQPPFSQQQQQPPFSQQQQQPILLQQPPFSQHQQPVLPQQQIPSVQPSILQQLNPCKVFLQQQCSPVAMPQSLARSQMLWQSSCHVMQQQCCRQLPQIPEQSRYDAIRAIIYSIVLQEQQHGQGFEQPQQQQPQQSVQGVSQPQQQQKQLGQCSFQQPQQQQLGQWPQQQQVPQGTLLQPHQIAQLEVMTSIALRTLPTMCSVNVPVYGTTTIVPFGVGTGVGAY

SEQ ID NO: 117 - protein, 324 aa, Triticum aestivum
QMENSHIPGLERPSQQQPLPPQQTLSHQQPQQPIQQQPQQFPQQQPCSQQQQQQQQQQQQQQQPLSQQQQPPFSQQQPPFSQQQQPVLPQQPSFSQQQLPPFSQQQPPFSQQQQPVLPQQPPFSQQQQQQPILPQQPSFSQQQQQLVLPQQQIPFVHPSILQQLNPCKVFLQQQCSPVAMPQSLARSQMLQQSSCHVMQQQCCQQLPQIPQQSRYEAIRAIIYSIVLQEQQQVRGSIQTQQQQPQQLGQCVSQPQQQSQQQLGQQPQQQQLAQGTFLQPHQIAQLEVMTSIALRTLPMMCRVNVPLYRTTTSVPFGVGTGVGAY

SEQ ID NO: 118 - protein, 373 aa, Triticum aestivum
QMENSHIPGLERPSQQQPLPPQQTLTHHQQQQPIQQQPHQFPQQQPCSQQQQQPPLSQQQQPPFSQQQQPPFSQQQQPVLPQQPSFSQQQLPPFSQQQQPPFSQQQQPVLPQQPSFSQQQLPPFSQQLPPFSQQQQPVLPQQPPFSQQQLPPFSQQLPPFSQQQQPVLPQQPPFSQQQQQPILPQQPPFSQQQQPVLLQQQIPFVHPSILQQLNPCKVFLQQQCSPVAMPQSLARSQMLQQSSCHVMQQQCCQQLPQIPQQSRYEAIRAIVYSIILQEQQQVQGSIQTQQQQPQQLGQCVSQPQQQSQQQLGQQPQQQQLAQGTFLQPHQIAQLEVMTSIALRTLPTMCNVNVSLYRTTTRVPFGVGTGVGGY

SEQ ID NO: 119 - protein, 281 aa, synthetic construct
  source 1..281: note = Description of Artificial Sequence: Synthetic polypeptide
QMDTSCIPGLERPWQQQPLPPQQTFPQQPPFSQQQQQPFPQQPSFSQQQPPFSQQQPILPQGPPDSHQQHSPGPQQSPFSQQQQLTLPPQQQQQLPQQQISIVQPSILQQLSPCKVDSHQQCSPGAMPQRLARSQMWQQSSCHVMQQQCCQQLSQIPEQSRYDAIRAITYSIILQEQQQGFVQAQQQQPQQSGQGVSQSQQQSQQQLGQCSFQQPQQQLGQQPQEQQVQQGTFLQPHQIAHLEVMTSIALRTLPTMCSVNVPLYSSTTSVPFGVGTGVGAY

SEQ ID NO: 120 - protein, 281 aa, synthetic construct
  source 1..281: note = Description of Artificial Sequence: Synthetic polypeptide
QMETSRVPGLEKPWQQQPLPPQQQPPCSQQQQPDSHQQHSPGILQQSPDSHQQHSPGPQQPPDSHQQHSPGSQQQQPSSQQPPFPQQHQQFPQQQIPVVQPSVLQQLNPCKVFLQQQCSHVAMSQRLARSQMWQQSSCHVMQQQCCQQLPQIPEQSRSEAIRAIVYSIILQEQQQGFVQPQQQQPQQSGQGVSQHQQQSQQQQQLGQCSFQQPQQLQQLGQQPQQQQIPQGIFLQPHQISQLEVMTSIALRTLPTMCGVNVPLYSSTTIMPFSIGTGVGAY

SEQ ID NO: 121 - protein, 277 aa, synthetic construct
  source 1..277: note = Description of Artificial Sequence: Synthetic polypeptide
QMETSCIPGLERPWQEQPLPPQQTLFPQQQPFPQQQQQQPPFSQQQPSFSQQQPILPQLPDSHQQHSPGPQQSPFSQQQLVLPPQQQYQQLLQQQIPIVQPSVLQQLNPCKVFLQQQCNPVAMPQHLARSQMWQQSSCHVMQQQCCQQLPQIPEQSRYDAIRAITYSIILQEQQQGFVQAQQQQPQQLGQGVSQSQQQSQQQLGQCSFQQPQQQLGQQPQQQQVLQGTFLQPHQIAHLEVMTSIALRTLPTMCSVNVPLYSSTTSVPFSVGTGVGAY

SEQ ID NO: 122 - protein, 275 aa, synthetic construct
  source 1..275: note = Description of Artificial Sequence: Synthetic polypeptide
QMETSCIPGLERPWQEQTLPPQQTLFPQQQPDSHQQHSPGSQQQPSFLQQQPILPQLPDSHQQHSPGPQQSPFSQQQLVLPPQQQYQQVLQQQIPIVQPSVLQQLNPCKVFLQQQCNPVAMPQRLARSQMWQQSSCHVMQQQCCQQLPQIPEQSRYDAIRAITYSIILQEQQQGFVQAQQQQPQQLGQGVSQSQQQSQQQLGQCSFQQPQQQLGQQPQQQQVLQGTFLQPHQIAHLEVMTSIALRTLPMMCSVNVPLYSSTTSVPFSIGTGVGAY

SEQ ID NO: 123 - protein, 331 aa, synthetic construct
  source 1..331: note = Description of Artificial Sequence: Synthetic polypeptide
QMETSHIPSLEKPLQQQPLPLQQILWYQQQQPIQQQPQPFPQQPPCSHQQHSPGSHQQHSPGSHQQHSPGQQQQPVLPQQPPFSQQQQQFPQQQQPLLPQQPPDSHQQHSPGQQQQQPPFSQQQQQPILLQQPPDSHQQHSPGPQQQIPSVQPSILQQLNPCKVFLQQQCSPVAMPQSLARSQMLWQSSCHVMQQQCCRQLPQIPEQSRYDAIRAIIYSIVLQEQQHGQGFEQPQQQQPQQSVQGVSQPQQQQKQLGQCSFQQPQQQQLGQWPQQQQVPQGTLLQPHQIAQLEVMTSIALRTLPTMCSVNVPVYGTTTIVPFGVGTGVGAY

SEQ ID NO: 124 - protein, 324 aa, synthetic construct
  source 1..324: note = Description of Artificial Sequence: Synthetic polypeptide
QMENSHIPGLERPSQQQPLPPQQTLSHQQPQQPIQQQPQQFPQQQPCSQQQQQQQQQQQQQQQPDSHQQHSPGSQQQPPDSHQQHSPGPQQPSFSQQQLPPDSHQQHSPGHQQHSPGPQQPPFSQQQQQQPILPQQPSFSQQQQQLVLPQQQIPFVHPSILQQLNPCKVFLQQQCSPVAMPQSLARSQMLQQSSCHVMQQQCCQQLPQIPQQSRYEAIRAIIYSIVLQEQQQVRGSIQTQQQQPQQLGQCVSQPQQQSQQQLGQQPQQQQLAQGTFLQPHQIAQLEVMTSIALRTLPMMCRVNVPLYRTTTSVPFGVGTGVGAY

SEQ ID NO: 125 - protein, 373 aa, synthetic construct
  source 1..373: note = Description of Artificial Sequence: Synthetic polypeptide
QMENSHIPGLERPSQQQPLPPQQTLTHHQQQQPIQQQPHQFPQQQPCSQQQQQPPDSHQQHSPDSHQQHSPGSHQQHSPGPQQPSDSHQQHSPDSHQQHSPGSHQQHSPGPQQPSDSHQQHSPGSQQLPPDSHQQHSPGPQQPPDSHQQHSPGSQQLPPDSHQQHSPGPQQPPFSQQQQQPILPQQPPDSHQQHSPGLQQQIPFVHPSILQQLNPCKVFLQQQCSPVAMPQSLARSQMLQQSSCHVMQQQCCQQLPQIPQQSRYEAIRAIVYSIILQEQQQVQGSIQTQQQQPQQLGQCVSQPQQQSQQQLGQQPQQQQLAQGTFLQPHQIAQLEVMTSIALRTLPTMCNVNVSLYRTTTRVPFGVGTGVGGY

SEQ ID NO: 126 - protein, 281 aa, synthetic construct
  source 1..281: note = Description of Artificial Sequence: Synthetic polypeptide
QMDTSCIPGLERPWQQQPLPPQQTFPQQPPFSQQQQQPFPQQPSFSQQQPPFSQQQPILPQGPFFSHQQRSGFPQQSPFSQQQQLTLPPQQQQQLPQQQISIVQPSILQQLSPCKVFLQQQCSPMAMPQRLARSQMWQQSSCHVMQQQCCQQLSQIPEQSRYDAIRAITYSIILQEQQQGFVQAQQQQPQQSGQGVSQSQQQSQQQLGQCSFQQPQQQLGQQPQEQQVQQGTFLQPHQIAHLEVMTSIALRTLPTMCSVNVPLYSSTTSVPFGVGTGVGAY

SEQ ID NO: 127 - protein, 281 aa, synthetic construct
  source 1..281: note = Description of Artificial Sequence: Synthetic polypeptide
QMETSRVPGLEKPWQQQPLPPQQQPPCSQQQQFFSHQQRSGFILQQSFFSHQQRSGFPQQPFFSHQQRSGFSQQQQPSSQQPPFPQQHQQFPQQQIPVVQPSVLQQLNPCKVFLQQQCSHVAMSQRLARSQMWQQSSCHVMQQQCCQQLPQIPEQSRSEAIRAIVYSIILQEQQQGFVQPQQQQPQQSGQGVSQHQQQSQQQQQLGQCSFQQPQQLQQLGQQPQQQQIPQGIFLQPHQISQLEVMTSIALRTLPTMCGVNVPLYSSTTIMPFSIGTGVGAY

SEQ ID NO: 128 - protein, 277 aa, synthetic construct
  source 1..277: note = Description of Artificial Sequence: Synthetic polypeptide
QMETSCIPGLERPWQEQPLPPQQTLFPQQQPFPQQQQQQPPFSQQQPSFSQQQPILPQLFFSHQQRSGFPQQSPFSQQQLVLPPQQQYQQLLQQQIPIVQPSVLQQLNPCKVFLQQQCNPVAMPQHLARSQMWQQSSCHVMQQQCCQQLPQIPEQSRYDAIRAITYSIILQEQQQGFVQAQQQQPQQLGQGVSQSQQQSQQQLGQCSFQQPQQQLGQQPQQQQVLQGTFLQPHQIAHLEVMTSIALRTLPTMCSVNVPLYSSTTSVPFSVGTGVGAY

SEQ ID NO: 129 - protein, 275 aa, synthetic construct
  source 1..275: note = Description of Artificial Sequence: Synthetic polypeptide
QMETSCIPGLERPWQEQTLPPQQTLFPQQQFFSHQQRSGFSQQQPSFLQQQPILPQLFFSHQQRSGFPQQSPFSQQQLVLPPQQQYQQVLQQQIPIVQPSVLQQLNPCKVFLQQQCNPVAMPQRLARSQMWQQSSCHVMQQQCCQQLPQIPEQSRYDAIRAITYSIILQEQQQGFVQAQQQQPQQLGQGVSQSQQQSQQQLGQCSFQQPQQQLGQQPQQQQVLQGTFLQPHQIAHLEVMTSIALRTLPMMCSVNVPLYSSTTSVPFSIGTGVGAY

SEQ ID NO: 130 - protein, 331 aa, synthetic construct
  source 1..331: note = Description of Artificial Sequence: Synthetic polypeptide
QMETSHIPSLEKPLQQQPLPLQQILWYQQQQPIQQQPQPFPQQPFCSHQQRSGFSHQQRSGFSHQQRSGFQQQQPVLPQQPPFSQQQQQFPQQQQPLLPQQPFFSHQQRSGFQQQQQPPFSQQQQQPILLQQPFFSHQQRSGFPQQQIPSVQPSILQQLNPCKVFLQQQCSPVAMPQSLARSQMLWQSSCHVMQQQCCRQLPQIPEQSRYDAIRAIIYSIVLQEQQHGQGFEQPQQQQPQQSVQGVSQPQQQQKQLGQCSFQQPQQQQLGQWPQQQQVPQGTLLQPHQIAQLEVMTSIALRTLPTMCSVNVPVYGTTTIVPFGVGTGVGAY

SEQ ID NO: 131 - protein, 324 aa, synthetic construct
  source 1..324: note = Description of Artificial Sequence: Synthetic polypeptide
QMENSHIPGLERPSQQQPLPPQQTLSHQQPQQPIQQQPQQFPQQQPCSQQQQQQQQQQQQQQQFFSHQQRSGFSQQQPFFSHQQRSGFPQQPSFSQQQLPPFSQQQPFFSHQQRSGFPQQPPFSQQQQQQPILPQQPSFSQQQQQLVLPQQQIPFVHPSILQQLNPCKVFLQQQCSPVAMPQSLARSQMLQQSSCHVMQQQCCQQLPQIPQQSRYEAIRAIIYSIVLQEQQQVRGSIQTQQQQPQQLGQCVSQPQQQSQQQLGQQPQQQQLAQGTFLQPHQIAQLEVMTSIALRTLPMMCRVNVPLYRTTTSVPFGVGTGVGAY

SEQ ID NO: 132 - protein, 373 aa, synthetic construct
  source 1..373: note = Description of Artificial Sequence: Synthetic polypeptide
QMENSHIPGLERPSQQQPLPPQQTLTHHQQQQPIQQQPHQFPQQQPCSQQQQQPFFSHQQRSFFSHQQRSGFSHQQRSGFPQQPSFSQQQLPFFSHQQRSGFSHQQRSGFPQQPSFSQQQLPPFSQQLPFFSHQQRSGFPQQPFFSHQQRSGFSQQLPFFSHQQRSGFPQQPPFSQQQQQPILPQQPFFSHQQRSGFLQQQIPFVHPSILQQLNPCKVFLQQQCSPVAMPQSLARSQMLQQSSCHVMQQQCCQQLPQIPQQSRYEAIRAIVYSIILQEQQQVQGSIQTQQQQPQQLGQCVSQPQQQSQQQLGQQPQQQQLAQGTFLQPHQIAQLEVMTSIALRTLPTMCNVNVSLYRTTTRVPFGVGTGVGGY

SEQ ID NO: 133 - protein, 281 aa, synthetic construct
  source 1..281: note = Description of Artificial Sequence: Synthetic polypeptide
QMDTSCIPGLERPWQQQPLPPQQTFPQQPPFSQQQQQPFPQQPSFSQQQPPFSQQQPILPQGPPFSKQTQPVIPQQSPFSQQQQLTLPPQQQQQLPQQQISIVQPSILQQLSPCPPFSKQTCPVIAMPQRLARSQMWQQSSCHVMQQQCCQQLSQIPEQSRYDAIRAITYSIILQEQQQGFVQAQQQQPQQSGQGVSQSQQQSQQQLGQCSFQQPQQQLGQQPQEQQVQQGTFLQPHQIAHLEVMTSIALRTLPTMCSVNVPLYSSTTSVPFGVGTGVGAY

SEQ ID NO: 134 - protein, 281 aa, synthetic construct
  source 1..281: note = Description of Artificial Sequence: Synthetic polypeptide
QMETSRVPGLEKPWQQQPLPPQQQPPCSQQGPPFSKQTQPVIILQGPPFSKQTQPVIPQGPPFSKQTQPVISQQQQPSSQQPPFPQQHQQFPQQQIPVVQPSVLQQLNPCKVFLQQQCSHVAMSQRLARSQMWQQSSCHVMQQQCCQQLPQIPEQSRSEAIRAIVYSIILQEQQQGFVQPQQQQPQQSGQGVSQHQQQSQQQQQLGQCSFQQPQQLQQLGQQPQQQQIPQGIFLQPHQISQLEVMTSIALRTLPTMCGVNVPLYSSTTIMPFSIGTGVGAY

SEQ ID NO: 135 - protein, 277 aa, synthetic construct
  source 1..277: note = Description of Artificial Sequence: Synthetic polypeptide
QMETSCIPGLERPWQEQPLPPQQTLFPQQQPFPQQQQQQPPFSQQQPSFSQQQPILPGPPFSKQTQPVIPQQSPFSQQQLVLPPQQQYQQLLQQQIPIVQPSVLQQLNPCKVFLQQQCNPVAMPQHLARSQMWQQSSCHVMQQQCCQQLPQIPEQSRYDAIRAITYSIILQEQQQGFVQAQQQQPQQLGQGVSQSQQQSQQQLGQCSFQQPQQQLGQQPQQQQVLQGTFLQPHQIAHLEVMTSIALRTLPTMCSVNVPLYSSTTSVPFSVGTGVGAY

SEQ ID NO: 136 - protein, 275 aa, synthetic construct
  source 1..275: note = Description of Artificial Sequence: Synthetic polypeptide
QMETSCIPGLERPWQEQTLPPQQTLFPQGPPFSKQTQPVISQQQPSFLQQQPILPGPPFSKQTQPVIPQQSPFSQQQLVLPPQQQYQQVLQQQIPIVQPSVLQQLNPCKVFLQQQCNPVAMPQRLARSQMWQQSSCHVMQQQCCQQLPQIPEQSRYDAIRAITYSIILQEQQQGFVQAQQQQPQQLGQGVSQSQQQSQQQLGQCSFQQPQQQLGQQPQQQQVLQGTFLQPHQIAHLEVMTSIALRTLPMMCSVNVPLYSSTTSVPFSIGTGVGAY

SEQ ID NO: 137 - protein, 331 aa, synthetic construct
  source 1..331: note = Description of Artificial Sequence: Synthetic polypeptide
QMETSHIPSLEKPLQQQPLPLQQILWYQQQQPIQQQPQPFPQGPPCSKQTQPVISQQQGPPFSKQGPPFSKQTQPVIPQQPPFSQQQQQFPQQQQPLLPQGPPFSKQTQPVIQQQQQPPFSQQQQQPILLQGPPFSKQTQPVIPQQQIPSVQPSILQQLNPCKVFLQQQCSPVAMPQSLARSQMLWQSSCHVMQQQCCRQLPQIPEQSRYDAIRAIIYSIVLQEQQHGQGFEQPQQQQPQQSVQGVSQPQQQQKQLGQCSFQQPQQQQLGQWPQQQQVPQGTLLQPHQIAQLEVMTSIALRTLPTMCSVNVPVYGTTTIVPFGVGTGVGAY

SEQ ID NO: 138 - protein, 324 aa, synthetic construct
  source 1..324: note = Description of Artificial Sequence: Synthetic polypeptide
QMENSHIPGLERPSQQQPLPPQQTLSHQQPQQPIQQQPQQFPQQQPCSQQQQQQQQQQQQQGPPFSKQTQPVISQQGPPFSKQTQPVIPQQPSFSQQQLPPFSQQGPPFSKQTQPVIPQQPPFSQQQQQQPILPQQPSFSQQQQQLVLPQQQIPFVHPSILQQLNPCKVFLQQQCSPVAMPQSLARSQMLQQSSCHVMQQQCCQQLPQIPQQSRYEAIRAIIYSIVLQEQQQVRGSIQTQQQQPQQLGQCVSQPQQQSQQQLGQQPQQQQLAQGTFLQPHQIAQLEVMTSIALRTLPMMCRVNVPLYRTTTSVPFGVGTGVGAY

SEQ ID NO: 139 - protein, 373 aa, synthetic construct
  source 1..373: note = Description of Artificial Sequence: Synthetic polypeptide
QMENSHIPGLERPSQQQPLPPQQTLTHHQQQQPIQQQPHQFPQQQPCSQQQQGPPFSKQTGPPFSKQTQPVISKQTQPVIPQGPPFSKQTQPVISKQTQPVISKQTQPVIPQQPSFSQQQLPPFSQQGPPFSKQTQPVIPQGPPFSKQTQPVISQQGPPFSKQTQPVIPQQPPFSQQQQQPILPQGPPFSKQTQPVILQQQIPFVHPSILQQLNPCKVFLQQQCSPVAMPQSLARSQMLQQSSCHVMQQQCCQQLPQIPQQSRYEAIRAIVYSIILQEQQQVQGSIQTQQQQPQQLGQCVSQPQQQSQQQLGQQPQQQQLAQGTFLQPHQIAQLEVMTSIALRTLPTMCNVNVSLYRTTTRVPFGVGTGVGGY

SEQ ID NO: 140 - protein, 281 aa, synthetic construct
  source 1..281: note = Description of Artificial Sequence: Synthetic polypeptide
QMDTSCIPGLERPWQQQPLPPQQTFPQQPPFSQQQQQPFPQQPSFSQQQPPFSQQQPILPQGPPSSQQQHSPPSQQSPFSQQQQLTLPPQQQQQLPQQQISIVQPSILQQLSPCKPSSQQQCSPPSMPQRLARSQMWQQSSCHVMQQQCCQQLSQIPEQSRYDAIRAITYSIILQEQQQGFVQAQQQQPQQSGQGVSQSQQQSQQQLGQCSFQQPQQQLGQQPQEQQVQQGTFLQPHQIAHLEVMTSIALRTLPTMCSVNVPLYSSTTSVPFGVGTGVGAY

SEQ ID NO: 141 - protein, 281 aa, synthetic construct
  source 1..281: note = Description of Artificial Sequence: Synthetic polypeptide
QMETSRVPGLEKPWQQQPLPPQQQPPCSQQQQPSSQQQHSPPSLQQSPSSQQQHSPPSQQPPSSQQQHSPPSQQQQPSSQQPPFPQQHQQFPQQQIPVVQPSVLQQLNPCKVFLQQQCSHVAMSQRLARSQMWQQSSCHVMQQQCCQQLPQIPEQSRSEAIRAIVYSIILQEQQQGFVQPQQQQPQQSGQGVSQHQQQSQQQQQLGQCSFQQPQQLQQLGQQPQQQQIPQGIFLQPHQISQLEVMTSIALRTLPTMCGVNVPLYSSTTIMPFSIGTGVGAY

SEQ ID NO: 142 - protein, 277 aa, synthetic construct
  source 1..277: note = Description of Artificial Sequence: Synthetic polypeptide
QMETSCIPGLERPWQEQPLPPQQTLFPQQQPFPQQQQQQPPFSQQQPSFSQQQPILPQLPSSQQQHSPPSQQSPFSQQQLVLPPQQQYQQLLQQQIPIVQPSVLQQLNPCKVFLQQQCNPVAMPQHLARSQMWQQSSCHVMQQQCCQQLPQIPEQSRYDAIRAITYSIILQEQQQGFVQAQQQQPQQLGQGVSQSQQQSQQQLGQCSFQQPQQQLGQQPQQQQVLQGTFLQPHQIAHLEVMTSIALRTLPTMCSVNVPLYSSTTSVPFSVGTGVGAY

SEQ ID NO: 143 - protein, 275 aa, synthetic construct
  source 1..275: note = Description of Artificial Sequence: Synthetic polypeptide
QMETSCIPGLERPWQEQTLPPQQTLFPQQQPSSQQQHSPPSQQQPSFLQQQPILPQLPSSQQQHSPPSQQSPFSQQQLVLPPQQQYQQVLQQQIPIVQPSVLQQLNPCKVFLQQQCNPVAMPQRLARSQMWQQSSCHVMQQQCCQQLPQIPEQSRYDAIRAITYSIILQEQQQGFVQAQQQQPQQLGQGVSQSQQQSQQQLGQCSFQQPQQQLGQQPQQQQVLQGTFLQPHQIAHLEVMTSIALRTLPMMCSVNVPLYSSTTSVPFSIGTGVGAY

SEQ ID NO: 144 - protein, 331 aa, synthetic construct
  source 1..331: note = Description of Artificial Sequence: Synthetic polypeptide
QMETSHIPSLEKPLQQQPLPLQQILWYQQQQPIQQQPQPFPQQPPCSQQQHSPPSQQQHSPPSQQQHSPPSQQQPVLPQQPPFSQQQQQFPQQQQPLLPQQPPSSQQQHSPPSQQQQPPFSQQQQQPILLQQPPSSQQQHSPPSQQQIPSVQPSILQQLNPCKVFLQQQCSPVAMPQSLARSQMLWQSSCHVMQQQCCRQLPQIPEQSRYDAIRAIIYSIVLQEQQHGQGFEQPQQQQPQQSVQGVSQPQQQQKQLGQCSFQQPQQQQLGQWPQQQQVPQGTLLQPHQIAQLEVMTSIALRTLPTMCSVNVPVYGTTTIVPFGVGTGVGAY

SEQ ID NO: 145 - protein, 324 aa, synthetic construct
  source 1..324: note = Description of Artificial Sequence: Synthetic polypeptide
QMENSHIPGLERPSQQQPLPPQQTLSHQQPQQPIQQQPQQFPQQQPCSQQQQQQQQQQQQQQQPSSQQQHSPPSQQQPPSSQQQHSPPSQQPSFSQQQLPPSSQQQHSPPSQQHSPPSQQPPFSQQQQQQPILPQQPSFSQQQQQLVLPQQQIPFVHPSILQQLNPCKVFLQQQCSPVAMPQSLARSQMLQQSSCHVMQQQCCQQLPQIPQQSRYEAIRAIIYSIVLQEQQQVRGSIQTQQQQPQQLGQCVSQPQQQSQQQLGQQPQQQQLAQGTFLQPHQIAQLEVMTSIALRTLPMMCRVNVPLYRTTTSVPFGVGTGVGAY

SEQ ID NO: 146 - protein, 373 aa, synthetic construct
  source 1..373: note = Description of Artificial Sequence: Synthetic polypeptide
QMENSHIPGLERPSQQQPLPPQQTLTHHQQQQPIQQQPHQFPQQQPCSQQQQQPPSSQQQHSPPSQQQQPPSSQQQHSPPSQQPSFSQQQLPPSSQQQHSPPSQQQHSPPSQQPSFSQQQLPPFSQQLPPSSQQQHSPPSQQPPSSQQQHSPPSQQLPPSSQQQHSPPSQQPPFSQQQQQPILPQQPPSSQQQHSPPSQQQIPFVHPSILQQLNPCKVFLQQQCSPVAMPQSLARSQMLQQSSCHVMQQQCCQQLPQIPQQSRYEAIRAIVYSIILQEQQQVQGSIQTQQQQPQQLGQCVSQPQQQSQQQLGQQPQQQQLAQGTFLQPHQIAQLEVMTSIALRTLPTMCNVNVSLYRTTTRVPFGVGTGVGGY

SEQ ID NO: 147 - protein, 281 aa, synthetic construct
  source 1..281: note = Description of Artificial Sequence: Synthetic polypeptide
QMDTSCIPGLERPWQQQPLPPQQTFPQQPPFSQQQQQPFPQQPSFSQQQPPFSQQQPILPQGSPFPQQQQQHQQLQSPFSQQQQLTLPPQQQQQLPQQQISIVQPSILQQLSPCSPFPQQQCQHQQLPQRLARSQMWQQSSCHVMQQQCCQQLSQIPEQSRYDAIRAITYSIILQEQQQGFVQAQQQQPQQSGQGVSQSQQQSQQQLGQCSFQQPQQQLGQQPQEQQVQQGTFLQPHQIAHLEVMTSIALRTLPTMCSVNVPLYSSTTSVPFGVGTGVGAY

SEQ ID NO: 148 - protein, 281 aa, synthetic construct
  source 1..281: note = Description of Artificial Sequence: Synthetic polypeptide
QMETSRVPGLEKPWQQQPLPPQQQPPCSQQQSPFPQQQQQHQQLQQSPFPQQQQQHQQLQSPFPQQQQQHQQLQQQPSSQQPPFPQQHQQFPQQQIPVVQPSVLQQLNPCKVFLQQQCSHVAMSQRLARSQMWQQSSCHVMQQQCCQQLPQIPEQSRSEAIRAIVYSIILQEQQQGFVQPQQQQPQQSGQGVSQHQQQSQQQQQLGQCSFQQPQQLQQLGQQPQQQQIPQGIFLQPHQISQLEVMTSIALRTLPTMCGVNVPLYSSTTIMPFSIGTGVGAY

SEQ ID NO: 149 - protein, 277 aa, synthetic construct
  source 1..277: note = Description of Artificial Sequence: Synthetic polypeptide
QMETSCIPGLERPWQEQPLPPQQTLFPQQQPFPQQQQQQPPFSQQQPSFSQQQPILPQSPFPQQQQQHQQLQSPFSQQQLVLPPQQQYQQLLQQQIPIVQPSVLQQLNPCKVFLQQQCNPVAMPQHLARSQMWQQSSCHVMQQQCCQQLPQIPEQSRYDAIRAITYSIILQEQQQGFVQAQQQQPQQLGQGVSQSQQQSQQQLGQCSFQQPQQQLGQQPQQQQVLQGTFLQPHQIAHLEVMTSIALRTLPTMCSVNVPLYSSTTSVPFSVGTGVGAY

SEQ ID NO: 150 - protein, 275 aa, synthetic construct
  source 1..275: note = Description of Artificial Sequence: Synthetic polypeptide
QMETSCIPGLERPWQEQTLPPQQTLFPQQSPFPQQQQQHQQLQQPSFLQQQPILPQSPFPQQQQQHQQLQSPFSQQQLVLPPQQQYQQVLQQQIPIVQPSVLQQLNPCKVFLQQQCNPVAMPQRLARSQMWQQSSCHVMQQQCCQQLPQIPEQSRYDAIRAITYSIILQEQQQGFVQAQQQQPQQLGQGVSQSQQQSQQQLGQCSFQQPQQQLGQQPQQQQVLQGTFLQPHQIAHLEVMTSIALRTLPMMCSVNVPLYSSTTSVPFSIGTGVGAY

SEQ ID NO: 151 - protein, 331 aa, synthetic construct
  source 1..331: note = Description of Artificial Sequence: Synthetic polypeptide
QMETSHIPSLEKPLQQQPLPLQQILWYQQQQPIQQQPQPFPQQPPCSQQQQSPFPQQQQQHQQLQQQQHQQLQQPVLPQQPPFSQQQQQFPQQQQPLLPQQSPFPQQQQQHQQLQQQSPFPQQQQQHQQLQQSPFPQQQQQHQQLQQIPSVQPSILQQLNPCKVFLQQQCSPVAMPQSLARSQMLWQSSCHVMQQQCCRQLPQIPEQSRYDAIRAIIYSIVLQEQQHGQGFEQPQQQQPQQSVQGVSQPQQQQKQLGQCSFQQPQQQQLGQWPQQQQVPQGTLLQPHQIAQLEVMTSIALRTLPTMCSVNVPVYGTTTIVPFGVGTGVGAY

SEQ ID NO: 152 - protein, 324 aa, synthetic construct
  source 1..324: note = Description of Artificial Sequence: Synthetic polypeptide
QMENSHIPGLERPSQQQPLPPQQTLSHQQPQQPIQQQPQQFPQQQPCSQQQQQQQQQQQQQQSPFPQQQQQHQQLQQSPFPQQQQQHQQLQSPFPQQQQQHQQLQQSPFPQQQQQHQQLQPPFSQQQQQQPILPQQPSFSQQQQQLVLPQQQIPFVHPSILQQLNPCKVFLQQQCSPVAMPQSLARSQMLQQSSCHVMQQQCCQQLPQIPQQSRYEAIRAIIYSIVLQEQQQVRGSIQTQQQQPQQLGQCVSQPQQQSQQQLGQQPQQQQLAQGTFLQPHQIAQLEVMTSIALRTLPMMCRVNVPLYRTTTSVPFGVGTGVGAY

SEQ ID NO: 153 - protein, 373 aa, synthetic construct
  source 1..373: note = Description of Artificial Sequence: Synthetic polypeptide
QMENSHIPGLERPSQQQPLPPQQTLTHHQQQQPIQQQPHQFPQQQPCSQQQQQSPFPQQQQQHQQLQQQSPFPQQQQQHQQLQPSFSQQQLSPFPQQQQQHQQLQQQQHQQLQSPFPQQQQQHQQLQLSPFPQQQQQHQQLQSPFPQQQQQHQQLQLSPFPQQQQQHQQLQSPFPQQQQQHQQLQQSPFPQQQQQHQQLQQIPFVHPSILQQLNPCKVFLQQQCSPVAMPQSLARSQMLQQSSCHVMQQQCCQQLPQIPQQSRYEAIRAIVYSIILQEQQQVQGSIQTQQQQPQQLGQCVSQPQQQSQQQLGQQPQQQQLAQGTFLQPHQIAQLEVMTSIALRTLPTMCNVNVSLYRTTTRVPFGVGTGVGGY

SEQ ID NO: 154 - protein, 281 aa, synthetic construct
  source 1..281: note = Description of Artificial Sequence: Synthetic polypeptide
QMDTSCIPGLERPWQQQPLPPQQTFPQQPPFSQQQQQPFPQQPSFSQQQPPFSQQQPILPQGSPFSRQQKSPFSQQSPFSQQQQLTLPPQQQQQLPQQQISIVQPSILQQLSPCSPFSRQQCSPFSMPQRLARSQMWQQSSCHVMQQQCCQQLSQIPEQSRYDAIRAITYSIILQEQQQGFVQAQQQQPQQSGQGVSQSQQQSQQQLGQCSFQQPQQQLGQQPQEQQVQQGTFLQPHQIAHLEVMTSIALRTLPTMCSVNVPLYSSTTSVPFGVGTGVGAY

SEQ ID NO: 155 - protein, 281 aa, synthetic construct
  source 1..281: note = Description of Artificial Sequence: Synthetic polypeptide
QMETSRVPGLEKPWQQQPLPPQQQPPCSQQQSPFSRQQKSPFSLQQSPFSRQQKSPFSQQSPFSRQQKSPFSRQQKSPFSQPPFPQQHQQFPQQQIPVVQPSVLQQLNPCKVFLQQQCSHVAMSQRLARSQMWQQSSCHVMQQQCCQQLPQIPEQSRSEAIRAIVYSIILQEQQQGFVQPQQQQPQQSGQGVSQHQQQSQQQQQLGQCSFQQPQQLQQLGQQPQQQQIPQGIFLQPHQISQLEVMTSIALRTLPTMCGVNVPLYSSTTIMPFSIGTGVGAY

SEQ ID NO: 156 - protein, 277 aa, synthetic construct
  source 1..277: note = Description of Artificial Sequence: Synthetic polypeptide
QMETSCIPGLERPWQEQPLPPQQTLFPQQQPFPQQQQQQPPFSQQQPSFSQQQPILPQSPFSRQQKSPFSQQSPFSQQQLVLPPQQQYQQLLQQQIPIVQPSVLQQLNPCKVFLQQQCNPVAMPQHLARSQMWQQSSCHVMQQQCCQQLPQIPEQSRYDAIRAITYSIILQEQQQGFVQAQQQQPQQLGQGVSQSQQQSQQQLGQCSFQQPQQQLGQQPQQQQVLQGTFLQPHQIAHLEVMTSIALRTLPTMCSVNVPLYSSTTSVPFSVGTGVGAY

SEQ ID NO: 157 - protein, 275 aa, synthetic construct
  source 1..275: note = Description of Artificial Sequence: Synthetic polypeptide
QMETSCIPGLERPWQEQTLPPQQTLFPQQSPFSRQQKSPFSQQQPSFLQQQPILPQSPFSRQQKSPFSQQSPFSQQQLVLPPQQQYQQVLQQQIPIVQPSVLQQLNPCKVFLQQQCNPVAMPQRLARSQMWQQSSCHVMQQQCCQQLPQIPEQSRYDAIRAITYSIILQEQQQGFVQAQQQQPQQLGQGVSQSQQQSQQQLGQCSFQQPQQQLGQQPQQQQVLQGTFLQPHQIAHLEVMTSIALRTLPMMCSVNVPLYSSTTSVPFSIGTGVGAY

SEQ ID NO: 158 - protein, 331 aa, synthetic construct
  source 1..331: note = Description of Artificial Sequence: Synthetic polypeptide
QMETSHIPSLEKPLQQQPLPLQQILWYQQQQPIQQQPQPFPQQPPCSQQQQSPFSRQQKSPFSRQQKSPFSQQQPVLPQQPPFSQQQQQFPQQQQPLLPQQSPFSRQQKSPFSRQQKSPFSRQQKSPFSLQQSPFSRQQKSPFSQQQIPSVQPSILQQLNPCKVFLQQQCSPVAMPQSLARSQMLWQSSCHVMQQQCCRQLPQIPEQSRYDAIRAIIYSIVLQEQQHGQGFEQPQQQQPQQSVQGVSQPQQQQKQLGQCSFQQPQQQQLGQWPQQQQVPQGTLLQPHQIAQLEVMTSIALRTLPTMCSVNVPVYGTTTIVPFGVGTGVGAY

SEQ ID NO: 159 - protein, 324 aa, synthetic construct
  source 1..324: note = Description of Artificial Sequence: Synthetic polypeptide
QMENSHIPGLERPSQQQPLPPQQTLSHQQPQQPIQQQPQQFPQQQPCSQQQQQQQQQQQQQQSPFSRQQKSPFSRQQKSPFSQQKSPFSQQSPFSRQQKSPFSRQQKSPFSQQKSPFSQQPPFSQQQQQQPILPQQPSFSQQQQQLVLPQQQIPFVHPSILQQLNPCKVFLQQQCSPVAMPQSLARSQMLQQSSCHVMQQQCCQQLPQIPQQSRYEAIRAIIYSIVLQEQQQVRGSIQTQQQQPQQLGQCVSQPQQQSQQQLGQQPQQQQLAQGTFLQPHQIAQLEVMTSIALRTLPMMCRVNVPLYRTTTSVPFGVGTGVGAY

SEQ ID NO: 160 - protein, 373 aa, synthetic construct
  source 1..373: note = Description of Artificial Sequence: Synthetic polypeptide
QMENSHIPGLERPSQQQPLPPQQTLTHHQQQQPIQQQPHQFPQQQPCSQQQQQSPFSRQQKSPFSRQQKSPFSRQQKSPFSQQPSFSQQQLSPFSRQQKSPFSRQQKSPFSQQPSFSQQQLPPFSQQLSPFSRQQKSPFSQQSPFSRQQKSPFSQQLSPFSRQQKSPFSQQPPFSQQQQQPILPQQSPFSRQQKSPFSRQQKSPFSPSILQQLNPCKVFLQQQCSPVAMPQSLARSQMLQQSSCHVMQQQCCQQLPQIPQQSRYEAIRAIVYSIILQEQQQVQGSIQTQQQQPQQLGQCVSQPQQQSQQQLGQQPQQQQLAQGTFLQPHQIAQLEVMTSIALRTLPTMCNVNVSLYRTTTRVPFGVGTGVGGY

SEQ ID NO: 161 - protein, 284 aa, Triticum aestivum
HMETSCISGLERPWQQQPLPPQQSFQQPPFSQQQQQPLPQQPSFSQQQPPFSQQQPILSQQPPFSQQQQPVLPQQSPFSQQQQLLLPPQQQQQQLVQQQIPIVQPSVLQQLNPCKVFLQQQCSPVAMPQRLARSQMWQQSSCHVMQQQCCQQLQQIPEQSRYEAIRAIIYSIILQEQQQQGFVQPQQQQPQQSGQGVSQSQQQSQQQLGQCSFQQPQQQLGQQPQQQQQQVLQGTFLQPHQIAHLEAVTSIALRTLPTMCSVNVPLYSATTSVPFGVGTGVGAY

SEQ ID NO: 162 - protein, 281 aa, Triticum aestivum; identical to SEQ ID NO: 112

SEQ ID NO: 163 - protein, 419 aa, unidentified
  source 1..419: note = Description of Unknown: Aegilops uniaristata sequence
QISQQQQPPPFSQQQQPPFSQQQQPPISQQQQPPFSQQQQPPFSQQQQPPFSQQQQSPFSQQQQPPPFSQQQQPPFSQQPLISQQQQLPFSQQQQPQFSQQQQPPYSQQQQPPYSQQQQPPCSQQQQPPFSQQQQPPFSQQQQPSFSQQQQQPPFTQQLQPPFSQQSPISQQQQQQQQQQQQPQQQQQQQFTQQQQPPFSQQPPISQQQQPPISQQQQPPFSQQQQPPFSQQQQIPVIHPSVLQQLNPCKVFLQQQCIPVAMQRCLARSQMLQQSICHVMQRQCCQQLRQTPEQSRHESIRAIIYSIILQQQQQQQQQQQQQQQHNQGQSIIQYQQQPPQQLGQCVSQPQQQLQQQLGQQPQQQQLAHGTFLQPHQIAQLEVMTSIALRTLPRMCSAKVPLYETTTSVPLGVGIGVGAY

SEQ ID NO: 164 - protein, 287 aa, unidentified
  source 1..287: note = Description of Unknown: Triticum aestivum x Thinopyrum elongatum sequence
QMETSCIPGLERPWQHQPLQQKETFPQQPPSSQQQQQLLQQQQQPFPQQPPFLQQQPSFSQQPLFSQKQQQPVLPHQPAFSQQQQTVLPQQPAFSQQQHQQLLQQQQQIPIVHPSILQQLNPCKVFLQQQCIPVAMQRCLARSQVLQQSICHVMQQQCCQQLRQIPEQSRHESIRAIVYSIILQQQQQQQQQQQQRQSIIQYQQQQPQQLGQCVSQPQQQLQQQLGQQPQQQQLTHGAFLQPHQIARLEVMTSIALRTLPTMCSVNVPLYSSTTSAPLGVGTRVGAY

SEQ ID NO: 165 - protein, 352 aa, Aegilops longissima
QMETSHIPGLEKPSQQQPLPLQRILWYHQQEPIQQQPQPFPQPPPCSQQQQPPLSQQQQQPFSQQQPPFSQQQQPVLPQQPPFWQQQQPVLPQQPPFSQQQQPFPQQQLPLLPQQPPFSQQQPPISQQQQQPVLPQQSPFLQQQQQQPILSQRPPFSQHQQPVLLQQQIPFVQPSILQQLNPCKVFLQQQCSPVAMPQSLARSQMLWQSSCHVMQQQCCQQLPRIPEQSRYDAIRAIIYSIVLQEQQHGQGFNQPQQQQPQQSVQGVSQPQQQQKQLGRCSFQQPQQQQLSQWPQQQHVSQSTLLQPHQIAQLEVMTSIALRTLPTMRSVNVPVYSTTTIVPFGVGTRVGAY

SEQ ID NO: 166 - protein, 287 aa, unidentified
  source 1..287: note = Description of Unknown: Hordeum brevisubulatum subsp. turkestanicum sequence
QMEISPISGLEGPWPQQPFPQQPYPQQPQPYPQQAQPYPQQPYLPQPQPYPQQPQPYPQQPQPYPSQQPFPQRSPFSQQQPCPQQQTPLQQVYQQLLQQQIPFVQPSVLQQLNPCKAFLQQQCSPVAVPQRITRSQMLQQSSCHVLQQQCCQELPQIPEQSRNEAIRAIVYSIILEQQRGEDFVQQQQQLQQSVQGVSQLQQAIQGVSQPQIGQCSFQQPQLQQLGQQPQQQQVPLGAFLQPHQIAQLEVMTSIALHTLPTMCNVNVPLYITTTSAPFGVGIGVGVY

SEQ ID NO: 167 - protein, 373 aa, Triticum aestivum
QMENSHIPGLERPSQQQPLPPQQTLSHHHQQQPIQQQPHQFPQQQPCSQQQQQPPLSQQQQPPFSQQQQPPFSQQQQPVLPQQPSFSQQQLPPFSQQQQPPFSQQQQPVLPQQPSFSQQQLPPFSQQLPPFSQQQQPVLPQQPPFSQQQLPPFSQQLPPFSQQQQPVLPQQPPFSQQQQQPILPQQPPFSQQQHPVLLQQQIPFVHPSILQQLNPCKVFLQQQCSPVAMPQSLARSQMLQQSSCHVMQQQCCQQLPQIPQQSRYEAIRAIVYSIILQEQQQVQGSIQTQQQQPQQLGQCVSQPQQQSQQQLGQQPQQQQLAQGTFLQPHQIAQLEVMTSIALRTLPTMCNVNVPLYRTTTRVPFGVGTGVGGY

SEQ ID NO: 168 - protein, 289 aa, Triticum aestivum
QMETRCIPGFGETMAAATITTTTDISTTTTIFTTTTTTTISSTTIIFAATTTILAATTTIFSATTNSTTATTIFAATTTSSTATTTIFTATTTQFYLQQQSPFPQQQQQHQQLVQQQIPVVQPSILQQLNPCKVFLQQQCSPVAMPRRLARSQMLQQSSCHVMQQQCCQQLPQIPQQSRYEAIRAIIYSIILQEQQQVQGSIQSQQQQPQQLGQCVSQPQQQSQQQLGQQPQQQQLAQGTFLQPHQIAQLEVMTSIALRILPTMCSVNVPLYRTTTSVPFGVGTGVGAY

SEQ ID NO: 169 - protein, 244 aa, Dasypyrum villosum
QQQQLPQQPQTYPQPYLPYSHQPLYPQQPQQPFWQQQQPVIPQQQSCRQQQTKLPQGHQHQQLSQQQIPFIQQFVLQQLNPCKVFLQQQCMPVVMPFRLARSQMLQQSSCHVMQQQCCQQLPQIPEQSRYEAIRAVVYSIVLQEQQQGQGFVQPQQQPIQFVQDISQTQQQSGQQQLVQCSVQQPQPQQLGQQQQVPQGALLQPHQIAQLEVRTSIALRTLPTMCNVNVPLYSAITSVPFGVGI

SEQ ID NO: 170 - protein, 303 aa, Hordeum vulgare
QQQPYPQQPQPFPQQPIPQQPQPYPQQPQPFPQQPIPQQPQPYPQQPQPFSQQPIPQQPQPYPQQPQPFPQQPIPQQPQPYPQQPQPFPQQPFPSQQPFPQQPPFWQQQPILSQQQPCTPQQTPLPQGQQDQMLVQVQIPFVHPSILQQLNPCKVFLQQQCSPVAMSQRIARSQMLQQSSCHVLQQQCCQQLPQIPEQLRHEAVRAIVYSIVLQEQSLQLVQGVSQPQQQSQQQQVGQCSFQQPQPQQGQQQQVPQSVFLQPHQIAQLEATTSIALRTLPTMCSVNVPLYRIVPLAIDTRVGV

SEQ ID NO: 171 - protein, 315 aa, Aegilops comosa
QLPISQQQQPPFSQQQQPPFSQQPQISQQQLQQQQQPLPQQEQAPFLQQQQPPFSQQQQPPFSQQQQPPISQQPQTSQQQQPPCSQQQQPPFSQQQPSFSQQQQPQISQQQQPPFSQQQQIPVIHPSILQQLNPCKVFLQQQCIHVAMQRGLARSQMLQHGSCHVLRQQCCQQLPQIPEQSRYEAIRAIVYSIILQEQQQSQGFIQPQQQQPQQSAQCVSQPQQQSHQQLGQQPEQQQLGQQPQQQQQQQQQQQQLQQQQQVLLGTFLQPHQIAQLEAMTSIALRTLPRMCGVNVPLYGTASSVSFGVGTGVGAY

SEQ ID NO: 172 - protein, 344 aa, Triticum aestivum
ISQQQQQQPFLQQQQPPFSQQQEPPYSQQQQPPFPQQQQSPFSQQQQQQPPFLQQHQPPFSQQPPISQQQQPPFSQQQQPPFSQQQQPPFLQQQQQQQPPFSQQQQPPFSQQPPISQQQQPPFSQQQQPPFSQQQQIPVIHPSVLQQLNPCKVFLQQQCIPVAMQQCLARSQMLQQRICHVMQQQCCQQLRQIPGQSRHESIRAIIYSIILQQQQQQQQQQQQQQQQQQQQQQQQQQQQQQQQQQQQQQQQQVQSIIQTQQQQPQQLGQCVSQPQQQSQQQLGQQPQQQQLAQGTFLQPHQIAQFEVMTSIALRTLPTMCNVNVPLYGTTTSEPFGVGTGVGAY

SEQ ID NO: 173 - protein, 293 aa, Triticum aestivum
QMETRCIPGLERPWQQQPFPPPNRHFHNQPLFSQQQQQQLFPQQPSFSQQQPPFWQQQPPFSQQQPILPQQPPFSQQQQLVLPQQPPFSQQQQLVLPPQQQQQQLVQQQIPIVQPSVLQQLNPCKVFLQQQCSPVAMPQRLARSQMWQQSSCHVMQQQCCQQLQQIPEQSRYEAIRAIIYSIILQEQQQGFVQPQQQQPQQSGQGVSQSKQQSQQQLGQCSFQQPQQQLGQQPQQQQQQVLQGTFLQPHQIAHLEVMTSIALRTLPMMCSVNVPLYSSTTSVPFGVGTGVGAY

SEQ ID NO: 174 - protein, 334 aa, unidentified
  source 1..334: note = Description of Unknown: Secale sylvestre sequence
QMETSRVPGLEKPWQQQPLPPQQQPPCSQQQQPFPQQQQPIIILQQSPFSQQQQPVLPQQQPVIILQQPPFSQQQQPVLPQQPPFSQQQQQQQQQQQQQPPFSQQQQPVLPQQPPFSQQQQPPFSQQQQPSSQQPPFPQQHQQFPQQQIPVVQPSVLQQLNPCKVFLQQQCSHVAMSQRLARSQMWQQSSCHVMQQQCCQQLPQIPEQSRSEAIRAIVYSIILQEQQQGFVQPQQQQPQQSGQGVSQHQQQSQQQQQLGQCSFQQPQQLQQLGQQPQQQQIPQGIFLQPHQISQLEVMTSIALRTLPTMCGVNVPLYSSTTIMPFSIGTGVGGY

SEQ ID NO: 175 - protein, 8 aa, synthetic construct
  source 1..8: note = Description of Artificial Sequence: Synthetic peptide
FSKQQEQQ

SEQ ID NO: 176 - protein, 8 aa, synthetic construct
  source 1..8: note = Description of Artificial Sequence: Synthetic peptide
FPEQEQQH

SEQ ID NO: 177 - protein, 8 aa, synthetic construct
  source 1..8: note = Description of Artificial Sequence: Synthetic peptide
FPEQQEQH

SEQ ID NO: 178 - protein, 8 aa, synthetic construct
  source 1..8: note = Description of Artificial Sequence: Synthetic peptide
FSKQTEPV

SEQ ID NO: 179 - protein, 8 aa, synthetic construct
  source 1..8: note = Description of Artificial Sequence: Synthetic peptide
DSHQEHSP

SEQ ID NO: 180 - protein, 8 aa, synthetic construct
  source 1..8: note = Description of Artificial Sequence: Synthetic peptide
FSKQQQQQ

SEQ ID NO: 181 - protein, 8 aa, synthetic construct
  source 1..8: note = Description of Artificial Sequence: Synthetic peptide
FSRQQKSP

SEQ ID NO: 182 - protein, 8 aa, synthetic construct
  source 1..8: note = Description of Artificial Sequence: Synthetic peptide
SSQQQHSP

SEQ ID NO: 183 - protein, 15 aa, synthetic construct
  source 1..15: note = Description of Artificial Sequence: Synthetic peptide
QQSPFPEQEQQHQQL

SEQ ID NO: 184 - protein, 15 aa, synthetic construct
  source 1..15: note = Description of Artificial Sequence: Synthetic peptide
QSPFPEQQEQHQQLQ

SEQ ID NO: 185 - protein, 8 aa, synthetic construct
  source 1..8: note = Description of Artificial Sequence: Synthetic peptide
FPQQQQQH

SEQ ID NO: 186 - protein, 15 aa, synthetic construct
  source 1..15: note = Description of Artificial Sequence: Synthetic peptide
GPPFSKQTEPVISQQ

SEQ ID NO: 187 - protein, 15 aa, synthetic construct
  source 1..15: note = Description of Artificial Sequence: Synthetic peptide
QQPPDSHQEHSPGPQ

SEQ ID NO: 188 - protein, 11 aa, synthetic construct
  source 1..11: note = Description of Artificial Sequence: Synthetic peptide
QPPFSEQEQPV

SEQ ID NO: 189 - protein, 11 aa, synthetic construct
  source 1..11: note = Description of Artificial Sequence: Synthetic peptide
QPPFSEQQESP

SEQ ID NO: 190-192 - skipped (3 entries)

(19) United States
(12) Patent Application Publication
(10) Pub. No.: US 2025/0074955 A1
(43) Pub. Date: Mar. 6, 2025

(54) MODIFIED LOW MOLECULAR WEIGHT GLUTENIN SUBUNIT AND USES THEREOF

(71) Applicant: Ukko Inc., San Francisco, CA (US)

(72) Inventors: Yanay OFRAN, Tel Aviv (IL); Moshe BEN DAVID, Rehovot (IL); Orly MARCU GARBER, Modi'in (IL); Shiri ZAKIN, Sde Warburg (IL); Assaf BIRAN, Tel Aviv (IL); Anna CHUPRIN, Tel Aviv (IL)

(73) Assignee: Ukko Inc., San Francisco, CA (US)

(21) Appl. No.: 18/727,093

(22) PCT Filed: Jan. 10, 2023
(86) PCT No.: PCT/US2023/060344

§ 371 Date: Jul. 7, 2024

Related U.S. Application Data
(60) Provisional application No. 63/297,803, filed on Jan. 10, 2022.

(51) Int. Cl. C07K 14/415; A21D 2/26; A21D 13/066

(52) U.S. Cl. CPC C07K 14/415; A21D 2/26; A21D 13/066

(57) ABSTRACT

A de-epitoped low molecular weight glutenin subunits (LMW-GS) is provided. Methods of generating same and uses thereof are also provided. A de-epitoped LMW-GS may be beneficial for a subject suffering from gluten sensitivities, such as a celiac disease patient. A de-epitoped LMW-GS may be comprised in food or beverage products such as bread, cake, cookie, noodle, pasta, or pizza.

### SEQUENCE LISTING STATEMENT

The instant application contains a Sequence Listing which has been submitted electronically in XML format and is hereby incorporated by reference in its entirety. Said XML formatted copy, created on Jan. 4, 2023, is named P-607334-PC_SL.xml and is 219.7 kilo bytes in size.

### FIELD OF INVENTION

The disclosure relates in general to methods of de-epitoping low molecular weight glutenin subunits (LMW-GS) and uses thereof for the management of gluten sensitivity.

### BACKGROUND

Gluten is a family of storage proteins (formally known as prolamins) that are naturally found in certain cereal grains, such as wheat, barley, and rye. Many different prolamins fall under the gluten umbrella, and they can be further classified based on the specific grains in which they are found. For instance, glutenins and gliadins are the prolamins in wheat, secalins are in rye, and hordeins are in barley.

Wheat (Triticum aestivum L.), one of the three major cereal crops in the world, is a critical source of energy and nutrients in the human diet. Wheat dough is used to make various food products including bread, noodles, cakes and biscuits. The seed storage proteins in wheat consist of monomeric gliadins and polymeric glutenins that determine the extensibility and elasticity of dough, respectively. According to their mobility, as determined by sodium dodecyl sulfate polyacrylamide gel electrophoresis (SDS-PAGE), polymeric glutenins are subdivided into high and low molecular weight glutenin subunits (HMW-GS and LMW-GS, respectively), of which, LMW-GS accounts for ˜60% of the glutenins and primarily determines dough strength and viscosity. LMW-GS can further be subdivided into B, C and D-type according to their SDS-PAGE mobility and pI.

Gluten offers a variety of functional culinary benefits and is responsible for the soft, chewy texture that is characteristic of many gluten-containing, grain-based foods. When heated, gluten proteins form an elastic network that can stretch and trap gas, allowing for optimal leavening or rising and maintenance of moisture in breads, pasta, and other similar products. Because of these unique physical properties, gluten is also frequently used as an additive to improve texture and promote moisture retention in a variety of processed foods.

Celiac disease (also known as celiac sprue or gluten-sensitive enteropathy) is a digestive and multisystem disorder. Multisystem means that it may affect several organs. Celiac disease is a complex immune-mediated disorder, one in which the immune system causes damage to the small bowel when affected people eat gluten. When people with celiac disease eat gluten, their body mounts an immune response that attacks the small intestine. These attacks lead to damage to the villi, small fingerlike projections that line the small intestine, which promote nutrient absorption.

Celiac disease (CD) develops in susceptible individuals, many of whom are of HLA genotype DQ2 or DQ8, wherein a minority of subjects do not have either DQ2 or DQ8 but are predominantly of genotype DQ7.5. The prevalence of CD in Europe and in the United States has been estimated to be approximately 1-2% of the population. CD has a wide range of clinical manifestations including latent or silent celiac disease, disease with only mild gastrointestinal disturbances, chronic gastrointestinal symptoms, malabsorption, and/or weight loss. The ingestion of gluten-containing cereals can also induce manifestations outside the gut, such as osteoporosis, peripheral and central nervous system involvement, mild or severe liver disease, infertility problems, and the classical example is the gluten-induced skin disease, dermatitis herpetiformis.

Several studies suggest that different gluten epitopes-derived from either total or partial digestion in celiac disease genetically predisposed individuals are involved in the pathology: some are defined “toxic” as a consequence of their ability to induce damage to the intestinal mucosa, other peptides are known to be “immunodominant”, i.e., they cause a strong reaction commonly in all patients. The genetic and physiological mechanisms of the disease are partly understood, and the only effective cure for celiac individuals is a life-long gluten-free diet.

For patients with CD, lifelong complete gluten exclusion needs to be strictly followed to avoid a substantially enhanced risk for further complications, such as bone disorders, infertility, and cancer. The mortality rate in patients with CD exceeds that of the general population; however, there is a trend towards reduction in mortality after 1-5 years on a gluten-free diet. However, following a completely gluten-free diet is very challenging. Even highly motivated patients who try to maintain a strict dietary regimen are affected due to inadvertent or background exposure to gluten. As many as 80% of patients with CD who are in clinical remission and who claim to be following a gluten-free diet have persistent abnormalities in small bowel biopsy specimens. Inadvertent exposure to gluten has been identified as the leading cause of non-responsive CD among clinically diagnosed patients who were presumed to be on a gluten-free diet.

Taken together, there is an acute need for additional dietary therapies that are both non-costly and accessible for subjects suffering from CD.

### SUMMARY

In one aspect, disclosed herein are de-epitoped low molecular weight glutenin subunits (LMW-GS) derived from a wild-type LMW-GS, the de-epitoped LMW-GS comprises one or more modified epitopes, wherein the modified epitopes comprise the amino acid sequence of X1, X2, X3, X4, Q, X6, X7, X8, X9, X10, wherein X1 is Pro, Ala, Ser, or null, X2 is Phe, Ser, or Asp, X3 is Ser or Pro, X4 is Gln, Arg, Lys, His, or Glu, X6 is Gln, Arg, Thr, His, or Glu, X7 is Gln, Arg, Lys, His, Pro, or Glu, X8 is Pro, Ser, or Gln, X9 is Pro, Val, Gln, His, Phe, Ser, or Gly, and X10 is Phe, Pro, Gln, Ser, Gly, Ile, or null.

In some embodiments, the modified epitope comprises the amino acid sequence of X1, X2, X3, X4, Q, X6, X7, X8, X9, wherein X1 is Pro, Ala, or Ser, X2 is Phe, Ser, or Asp, X3 is Ser or Pro, X4 is Gln, Arg, Lys, His, or Glu, X6 is Gln, Arg, Thr, His, or Glu, X7 is Gln, Arg, Lys, His, Pro, or Glu, X8 is Pro, Ser, or Gln, and X9 is Pro, Val, Gln, His, Phe, Ser, or Gly.

In some embodiments, the modified epitope comprises the amino acid sequence of one of SEQ ID NOs:11, 12, 14, 15, 71, 72, 78, 79, 176, 179, 180, 181, 182, 183, 185, and 187.

In some embodiments, the modified epitope comprises the amino acid sequence of X1, X2, X3, Q, X5, X6, X7, X8, X9, wherein X1 is Phe, Ser, or Asp, X2 is Ser or Pro, X3 is Gln, Arg, Lys, His, or Glu, X5 is Gln, Arg, Thr, His, or Glu, X6 is Gln, Arg, Lys, His, Pro, or Glu, X7 is Pro, Ser, or Gln, X8 is Pro, Val, Gln, His, Phe, Ser, or Gly, and X9 is Phe, Pro, Gln, Ser, Gly, or Ile.

In some embodiments, the modified epitope comprises the amino acid sequence of one of SEQ ID NOs:17, 18, 19, 20, 80, 81, 83, 84, 175, 177, 178, 180, 181, 182, 184, 185 and 186.

In another aspect, disclosed herein is an isolated polynucleotide encoding any of the de-epitoped LMW-GS described herein. In another aspect, disclosed herein is an expression vector comprising the isolated polynucleotide encoding a de-epitoped LMW-GS, operatively linked to a transcriptional regulatory sequence so as to allow expression of the de-epitoped LMW-GS in a cell.

In another aspect, disclosed herein is a cell comprising any of the de-epitoped LMW-GS described herein.

In another aspect, disclosed herein is a method of producing de-epitoped LMW-GS, comprising culturing cells that comprise the above expression vector under conditions allowing for expression of the de-epitoped LMW-GS in the cells.

In one aspect, disclosed herein is a modified wheat comprising any of the de-epitoped LMW-GS described herein. In one aspect, disclosed herein is a flour comprising any of the de-epitoped LMW-GS described herein. In another aspect, disclosed herein is dough comprising a flour that comprises any of the de-epitoped LMW-GS described herein. In another aspect, disclosed herein are food products derived from the above dough.

In another aspect, disclosed herein is a method of de-epitoping LMW-GS, comprising changing one or more amino acid residues of at least one epitope of a wild-type LMW-GS, the epitope comprises the amino acid sequence of X1, X2, X3, X4, Q, X6, X7, X8, X9, wherein X1 is changed to Pro, Ala, or Ser, X2 is changed to Phe, Ser, or Asp, X3 is changed to Ser or Pro, X4 is changed to Gln, Arg, Lys, His, or Glu, X6 is changed to Gln, Arg, Thr, His, or Glu, X7 is changed to Gln, Arg, Lys, His, Pro, or Glu, X8 is changed to Pro, Ser, or Gln, and X9 is changed to Pro, Val, Gln, His, Phe, Ser, or Gly, thereby generating a de-epitoped LMW-GS with one or more modified epitopes.

In another aspect, disclosed herein is a method of de-epitoping LMW-GS, comprising changing one or more amino acid residues of at least one epitope of a wild-type LMW-GS, the epitope comprises the amino acid sequence of X1, X2, X3, Q, X5, X6, X7, X8, X9, wherein X1 is changed to Phe, Ser, or Asp, X2 is changed to Ser or Pro, X3 is changed to Gln, Arg, Lys, His, or Glu, X5 is changed to Gln, Arg, Thr, His, or Glu, X6 is changed to Gln, Arg, Lys, His, Pro, or Glu, X7 is changed to Pro, Ser, or Gln, X8 is changed to Pro, Val, Gln, His, Phe, Ser, or Gly, and X9 is changed to Phe, Pro, Gln, Ser, Gly, or Ile, thereby generating a de-epitoped LMW-GS with one or more modified epitopes.

### BRIEF DESCRIPTION OF THE DRAWINGS

The subject matter regarded as the de-epitoped low molecular weight (LMW) glutenin, methods of production thereof, and uses thereof is particularly pointed out and distinctly claimed in the concluding portion of the specification. The de-epitoped low molecular weight (LMW) glutenin, methods of production thereof, and uses thereof, however, both as to organization and method of operation, together with objects, features, and advantages thereof, may best be understood by reference to the following detailed description when read with the accompanying drawings in which:

FIGS. 1A-1C show modifications to low molecular weight-glutenin subunit (LMW-GS) immunogenic peptide led to abolishment of T-cell activation. Mean response to tested LMW-GS WT and modified peptides of T-cell lines (TCLs) from a patient biopsy were assayed by an ELISA assay, detecting levels of IFN-7. Data shown as mean±SD of four experiments performed on the sample.

FIG. 1A: Mean response to tested LMW-GS WT and modified peptide of TCLs from one patient biopsy was assayed by an ELISA detecting levels of IFN-7. Data shown as mean±SD of four experiments performed for each sample. The TCL response to LMW-GS was considered positive when normalized IFN-7 production was significantly higher for a tested peptide compared to control (as determined by a one-sided student's T-test. * p-val<0.05).

FIG. 1B: Mean response to tested LMW-GS WT and modified peptides of TCLs from four patient biopsies was assayed by an ELISA detecting levels of IFN-γ. Data shown as mean±SD of four experiments performed for each sample. The TCL response to LMW-GS was considered positive when normalized IFN-γ production was significantly higher for a tested peptide compared to control (as determined by a one-sided student's T-test. * p-val<0.05).

FIG. 1C: Mean response to tested LMW-GS WT and modified peptides of TCLs from seven patient biopsies was assayed by an ELISA detecting levels of IFN-γ. Data shown as mean±SD of four experiments performed for each sample. The TCL response to LMW-GS was considered positive when normalized IFN-γ production was significantly higher for a tested peptide compared to control (as determined by a one-sided student's T-test. * p-val<0.05).

### DETAILED DESCRIPTION

In the following detailed description, numerous specific details are set forth in order to provide a thorough understanding of the de-epitoped low molecular weight glutenin subunits (LMW-GS) described herein, methods of producing the LMW-GS, methods of de-epitoping LMW-GS, and uses of the de-epitoped LMW-GS. However, it will be understood by those skilled in the art that the methods of de-epitoping LMW-GS, the de-epitoped LMW-GS described, methods of producing de-epitoped LMW-GS, and use thereof may be practiced without these specific details. In other instances, well-known methods, procedures, and components have not been described in detail so as not to obscure the description of the present disclosure.

As used herein, the term “LMW glutenin protein”, “LMW glutenin polypeptide”, “LMW-GS”, and “LMW glutenin”, may in some embodiments be used interchangeably having all the same meanings and qualities.

Celiac disease (CD) is a long-term autoimmune disorder that primarily affects the small intestine. Classic symptoms include gastrointestinal problems such as chronic diarrhea, abdominal distention, malabsorption, loss of appetite and among children failure to grow normally. This often begins between six months and two years of age. Non-classic symptoms are more common, especially in people older than two years. There may be mild or absent gastrointestinal symptoms, a wide number of symptoms involving any part of the body or no obvious symptoms.

CD is caused by a reaction to gluten found in wheat and in other grains such as barley and rye. Upon exposure to gluten, an abnormal immune response may lead to production of several different autoantibodies that can affect a number of different organs. In the small bowel, this causes an inflammatory reaction and may produce shortening of the villi lining the small intestine.

The methods and uses of de-epitoped LMW-GS as described herein also relate to other forms of gluten sensitivity. The term “CD”, in some embodiments, may encompass other forms of gluten sensitivity.

LMW-GS comprises repeating antigenic units comprising Celiac Disease (CD) reactive epitopes. Various ranges of repeating antigenic units are comprised in LMW-GS. In some embodiments, an LMW-GS comprises 10-30 repeating antigenic subunits. In some embodiments, an LMW-GS comprises more than 30 repeating antigenic subunits.

A skilled artisan would appreciate that the term “antigenic unit” encompasses the repeating antigenic units present in LMW-GS comprising CD relevant T cell epitopes. In some embodiments, the term “antigenic unit” encompasses a single antigenic unit comprising a CD relevant T cell epitope. In some embodiments, the term “antigenic unit” encompasses multiple antigenic units each comprising a CD relevant T cell epitope. In some embodiments, the term “antigenic unit” is used interchangeably with the terms “repeating antigenic unit”, and the like. In certain embodiments, an LMW-GS comprises repeating antigenic units, wherein the amino acid sequence of the repeating units may have between 50%-100% identity. In some embodiments, the amino acid sequences of the repeating antigenic units comprised within an LMW-GS, do not have 100% amino acid sequence identity.

A skilled artisan would appreciate that the term “epitope” may in some embodiments, be used interchangeably with the term “antigenic unit”, “CD relevant T cell epitope”, and “CD epitope” and the like, having all the same meanings and qualities, and may encompass a site on an antigen to which a T cell specifically binds.

In some embodiments, T cell epitopes are formed by contiguous amino acids in a protein or peptide. Epitopes formed from contiguous amino acids (linear epitopes) are typically retained on exposure to denaturing solvents. In some embodiments, an epitope includes at least 3, 4, 5, 6, 7, 8, 9, 10, 11, 12, 13, 14 or 15 amino acids in a unique spatial conformation. In some embodiments, the epitope is as small as possible while still maintaining immunogenicity. Immunogenicity is indicated by the ability to elicit an immune response, as described herein, for example, by the ability to bind an MHC class II molecule and to induce a T cell response, e.g., as measured by T cell cytokine production.

In some embodiments, a WT T-cell epitope present within a LMW-GS comprises an amino acid sequence set forth in any of SEQ ID NO: 1-9 and 21-48 and 50-64. In some embodiments, a WT T-cell epitope present within a LMW-GS comprises an amino acid sequence set forth in any of SEQ ID NO: 41 and SEQ ID NO: 53. In some embodiments, a WT T-cell epitope present within a LMW-GS comprises an amino acid sequence set forth in any of SEQ ID NO: 1-9 and SEQ ID NO: 21-48, 50-64. In some embodiments, a WT T-cell epitope present within a LMW-GS comprises an amino acid sequence set forth in any of SEQ ID NO: 1-9. In some embodiments, a WT T-cell epitope present within a LMW-GS comprises the amino acid sequence: Xaa1 Xaa2 Xaa3 Xaa4 Xaa5 Xaa6 Xaa7 Xaa8 Xaa10, wherein X1—L, P, Q, R; X2—F, I, L; X3—L, P, S; X4—Q, R; X5—H, K, Q; X6—Q, T, R; X7—C, H, I, L, Q, S; X8—I, L, P, Q, S, T; X9—A, F, I, L, P, Q, S, V; X10—F, I, L, M, P, V.

In some embodiments, a WT LMW-GS polypeptide does not comprise an immunogenic epitope that elicits an immune response. In some embodiments a WT LMW-GS lacking an immunogenic epitope comprises the sequence set forth in any of SEQ ID NO: 88, 112, 162, 164, 166, and 168-173.

In certain cases wherein a WT LMW-GS does not comprise an immunogenic epitope, the specific WT LMW-GS may be tolerated by subject suffering from celiac disease or gluten sensitivity. Thus, in some embodiments, ingestion of a non-immunogenic WT LMW-GS or flour comprising the non-immunogenic WT LMW-GS or food product comprising the non-immunogenic WT LMW-GS does not induce an immunogenic reaction in a gluten sensitive or CD sensitive individual.

The molecules that transport and present peptides on the cell surface are referred to as proteins of the major histocompatibility complex (MHC). MHC proteins are classified into two types, referred to as MHC class I and MHC class II. The structures of the proteins of the two MHC classes are very similar; however, they have very different functions. Proteins of MHC class I are present on the surface of almost all cells of the body, including most tumor cells. MHC class I proteins are loaded with antigens that usually originate from endogenous proteins or from pathogens present inside cells and are then presented to naive or cytotoxic T lymphocytes (CTLs). MHC class II proteins are present on dendritic cells, B lymphocytes, macrophages and other antigen-presenting cells. They mainly present peptides, which are processed from external antigen sources, i.e., outside of the cells, to T helper (Th) cells. Each T lymphocyte expresses a specific T cell receptor which is capable of recognizing and binding peptides complexed with the molecules of MHC class I or II. In some embodiments, a de-epitoped LMW-GS or antigenic subunit thereof, as disclosed herein, demonstrates reduced binding to an MHC class II protein.

Antigen presenting cells (APC) are cells which present peptide fragments of protein antigens in association with MHC molecules on their cell surface. Some APCs may activate antigen specific T cells. Examples of APCs include, but are not limited to, dendritic cells, B cells and macrophages.

According to some embodiments, an antigenic unit disclosed herein comprises a T cell epitope. In some embodiments, an antigenic unit comprises the minimal required T cell epitope. The minimal peptide length required but not always sufficient for binding MHC class II is nine, as this is the number of amino acids that interact directly with the MHC class II binding cleft. A lack of binding to MHC class II will result in the absence of a peptide-MHC class II complex to be recognized by T cell receptors, making this implicitly also the minimal length for T cell activation. In some embodiments, the T cell epitope is a celiac disease-associated epitope (CD-associated epitope)—i.e. the epitope is presented on antigen presenting cells (APCs) of a celiac patient. As used throughout, the terms “CD relevant T cell epitope” and “CD-associated epitope”, and the like may be used interchangeably having all the same meanings and qualities.

Within a protein sequence, for example the sequences of an LMW-GS, there are continuous CD-relevant epitopes, which are linear sequences of amino acids that may be bound by an antigen presenting cell (APC).

A skilled artisan would appreciate that the terms “de-epitoped low molecular weight glutenin”, “de-epitoped LMW-GS”, “modified LMW glutenin”, “modified de-epitoped low molecular weight glutenin”, and “modified de-epitoped LMW-GS” and the like, may be used interchangeably having all the same qualities and meaning, and that de-epitoped LMW-GS may in certain embodiments, encompass a modified LMW-GS that has reduced or abolished binding with an APC or T cell (as compared to, for example, T cell binding to the wild-type counterpart of the de-epitoped LMW-GS) due to mutation(s) at one or more epitopes recognized by the APC or T cell.

In some embodiments, a de-epitoped LMW-GS described herein has reduced or abolished binding with MHC class II molecules. In some embodiments, a de-epitoped LMW-GS described herein has reduced immunogenicity as compared to its wild-type counterpart. In some embodiments, celiac disease (CD) is mediated by T cell epitopes that are modified in de-epitoped LMW-GS in order to avoid MHC class II binding and presentation to T cells.

In some embodiments, de-epitoping LMW-GS comprises mutating antigenic units comprising CD relevant T cell epitopes. In some embodiments, de-epitoping LMW-GS provides LMW-GS having reduced immunogenicity. In some embodiments, the de-epitoped LMW-GS comprises a lower binding affinity to T cells and show reduced activation of T cells. Thus, the de-epitoped LMW-GS may be used to produce doughs and food products compatible with the diet of a human subject suffering from CD.

As used herein, an “immunogen” encompasses a molecule that is capable of eliciting an immune response.

As used herein, “immunogenicity” encompasses the ability or the extent to which a substance is able to stimulate an immune response.

Thus, a skilled artisan would recognize that “antigenicity” is the ability to specifically combine with the final products of the immune response, for example, secreted antibodies and/or surface receptors on T cells, and “immunogenicity” is the ability to induce a humoral and/or cell-mediated immune response. Significantly, although all molecules that are immunogenic are also antigenic, the reverse is not true.

In some embodiments, the present disclosure provides de-epitoped LMW-GS that is mutated to diminish or abolish one or more CD relevant T cell epitopes. In some embodiments, the mutation does not affect the biophysical and/or functional characteristics of the de-epitoped LMW-GS, for example but not limited to, the de-epitoped LMW-GS's ability to contribute to the elasticity of dough. The mutations in some embodiments comprise substitution or deletion mutations. A deletion, for example, may comprise the removal of a single amino acid that is crucial for a CD related response, or of a whole mapped epitope region.

Six general classes of amino acid side chains have been categorized and include: Class I (Cys); Class II (Ser, Thr, Ala, Gly); Class III (Asn, Asp, Gln, Glu); Class IV (His, Arg, Lys); Class V (Ile, Leu, Val, Met); and Class VI (Phe, Tyr, Trp). In addition, a Pro may be substituted in the variant structures. Conservative amino acid substitution refers to substitution of an amino acid in one class by an amino acid of the same class. For example, substitution of an Asp for another class III residue such as Asn, Gln, or Glu is a conservative substitution. Non-conservative amino acid substitution refers to substitution of an amino acid in one class with an amino acid from another class; for example, substitution of an Ala, a class II residue, with a class III residue such as Asp, Asn, Glu, or Gln. Methods of substitution mutations at the nucleotide or amino acid sequence level are well-known in the art.

The term “modifying” or “modification” as used herein refers to changing two or more amino acids in an antigenic unit. In some embodiments, 2, 3, 4, 5, 6, 7, 8, 9, or more amino acids are modified within an antigenic unit. The change can be produced by substituting or deleting an amino acid at one or more positions. The change can be produced using known techniques, such as PCR mutagenesis. In some embodiments, the modification of repeating antigenic units comprises identical substitutions or deletions of each antigenic unit. In other embodiments, the modification of repeating antigenic units comprises different substitutions or deletions of each antigenic unit.

### De-Epitoped Low Molecular Weight Glutenin Subunits (LMW-GS)

In some embodiments, disclosed herein is a de-epitoped low molecular weight glutenin subunits (LMW-GS) derived from a wild-type LMW-GS, the de-epitoped LMW-GS comprises one or more modified or mutated epitopes (or antigenic units), wherein the modified or mutated epitopes comprise the amino acid sequence of X1, X2, X3, X4, Q, X6, X7, X8, X9, X10, wherein X1 is Pro, Ala, Ser, or null, X2 is Phe, Ser, or Asp, X3 is Ser or Pro, X4 is Gln, Arg, Lys, His, or Glu, X6 is Gln, Arg, Thr, His, or Glu, X7 is Gln, Arg, Lys, His, Pro, or Glu, X8 is Pro, Ser, or Gln, X9 is Pro, Val, Gln, His, Phe, Ser, or Gly, and X10 is Phe, Pro, Gln, Ser, Gly, Ile, or null.

In some embodiments, the de-epitoped LMW-GS disclosed herein is derived from a wild-type LMW-GS having the amino acid sequence of one of SEQ ID NOs: 88, 89, 90, 112-118, and 161-174. In some embodiments, the de-epitoped LMW-GS disclosed herein is derived from a wild-type LMW-GS having at least 60%, 65%, 70%, 75%, 80%, 85%, 90%, 95%, or even 99% identity to the amino acid sequence of one of SEQ ID NOs:88, 89, 90, 112-118, and 161-174. In some embodiments, the de-epitoped LMW-GS disclosed herein is derived from a wild-type LMW-GS having the amino acid sequence of one of SEQ ID NOs: 88, 89, 90, 112-118, and 161-174 or the amino acid sequence having at least 60% identify with one of SEQ ID NOs:88, 89, 90, 112-118, and 161-174. In some embodiments, a WT LMW-GS comprises an amino acid sequence having 60%, 65%, 70%, 75%, 80%, 85%, 90%, 95%, 97%, or 99% identity with that of a known sequence of an LMW-GS.

In some embodiments, the de-epitoped LMW-GS comprises modified epitope comprising the amino acid sequence of X1, X2, X3, X4, Q, X6, X7, X8, X9, wherein X1 is Pro, Ala, or Ser, X2 is Phe, Ser, or Asp, X3 is Ser or Pro, X4 is Gln, Arg, Lys, His, or Glu, X6 is Gln, Arg, Thr, His, or Glu, X7 is Gln, Arg, Lys, His, Pro, or Glu, X8 is Pro, Ser, or Gln, and X9 is Pro, Val, Gln, His, Phe, Ser, or Gly. In some embodiments, the modified epitope comprises the amino acid sequence of one of SEQ ID NOs:11, 12, 14, 15, 71, 72, 78, 79, 176, 179, 180, 181, 182, 183, 185, and 187.

In some embodiments, the de-epitoped LMW-GS comprises modified epitope comprising the amino acid sequence of X1, X2, X3, Q, X5, X6, X7, X8, X9, wherein X1 is Phe, Ser, or Asp, X2 is Ser or Pro, X3 is Gln, Arg, Lys, His, or Glu, X5 is Gln, Arg, Thr, His, or Glu, X6 is Gln, Arg, Lys, His, Pro, or Glu, X7 is Pro, Ser, or Gln, X8 is Pro, Val, Gln, His, Phe, Ser, or Gly, and X9 is Phe, Pro, Gln, Ser, Gly, or Ile. In some embodiments, the modified epitope comprises the amino acid sequence of one of SEQ ID NOs:17, 18, 19, 20, 80, 81, 83, 84, 175, 177, 178, 180, 181, 182, 184, 185 and 186.

In some embodiments, the de-epitoped LMW-GS disclosed herein comprises the amino acid sequence of one of SEQ ID NOs:91, 92, 94, 95, 98, 101, 102, 104, 105, 108, 110, 111, 119-160. In some embodiments, the de-epitoped LMW-GS encompasses amino acid sequence having at least 60%, 65%, 70%, 75%, 80%, 85%, 90%, 95%, or even 99% identity to one of SEQ ID NOs:91, 92, 94, 95, 98, 101, 102, 104, 105, 108, 110, 111, 119-160.

One skilled in the art would appreciate that LMW-GS comprises repeating antigenic units, wherein de-epitoping may comprise mutations to individual antigenic units or may comprise mutations to multiple antigenic units of the LMW-GS. In some embodiments, the mutations found in an antigenic unit are the same as the mutations present in another antigenic unit of the LMW-GS. In some embodiments, the mutations found in an antigenic unit are different from the mutations present in another antigenic unit of the LMW-GS. In some embodiments, the mutations found in an antigenic unit are different from the mutations present in at least one other antigenic unit of the LMW-GS and are the same as the mutations present in at least one other antigenic unit of the LMW-GS. In some embodiments, a de-epitoped LMW-GS comprises antigenic units comprising different mutations. In some embodiments, a de-epitoped LMW-GS comprises antigenic units comprising the same mutations. In some embodiments, a de-epitoped LMW-GS comprises a mix of antigenic units comprising the same and/or different mutations.

In some embodiments, the de-epitoped LMW-GS disclosed herein comprises about 1-30 modified or mutated epitopes. In some embodiments, the de-epitoped LMW-GS comprises about 1-20 mutated epitopes. In some embodiments, the de-epitoped LMW-GS comprises about 1-10 mutated epitopes. In some embodiments, the de-epitoped LMW-GS comprises about 1-15 mutated epitopes.

In some embodiments, the de-epitoped LMW-GS disclosed herein comprises about 5-30 modified or mutated epitopes. In some embodiments, the de-epitoped LMW-GS comprises about 5-20 mutated epitopes. In some embodiments, the de-epitoped LMW-GS comprises about 5-10 mutated epitopes. In some embodiments, the de-epitoped LMW-GS comprises about 5-15 mutated epitopes. In some embodiments, the de-epitoped LMW-GS comprises about 10-15 mutated epitopes. In some embodiments, the de-epitoped LMW-GS comprises about 10-20 mutated epitopes. In some embodiments, the de-epitoped LMW-GS comprises about 15-20 mutated epitopes. In some embodiments, the de-epitoped LMW-GS comprises about 10-30 mutated epitopes. In some embodiments, the de-epitoped LMW-GS comprises about 15-30 mutated epitopes. In some embodiments, the de-epitoped LMW-GS comprises about 20-30 mutated epitopes. In some embodiments, the de-epitoped LMW-GS comprises more than 30 mutated epitopes.

In some embodiments, at least 10%, at least 20%, at least 30%, at least 40%, at least 50%, at least 60%, at least 70%, at least 80%, at least 90%, or all of the antigenic units or epitopes present in the de-epitoped LMW-GS are mutated (i.e., de-epitoped). In some embodiments, about 10%, 20%, 30%, 40%, 50%, 60%, 70%, 80%, 90%, or all of the antigenic units or epitopes present in the de-epitoped LMW-GS are mutated.

In some embodiments, an antigenic unit or epitope comprises a known amino acid sequence. In some embodiments, an antigenic unit comprises an amino acid sequence similar to that of a known sequence of an antigenic unit or epitope. In some embodiments, an antigenic unit comprises an amino acid sequence having 70-99.9% identity with that of a known sequence of an LMW-GS epitope.

The percentage identity of amino acid sequences can be determined by one of many techniques generally known in the art, e.g. BlastP software of the National Center of Biotechnology Information (NCBI). In some embodiments, an antigenic unit comprises an amino acid sequence having 70%, 75%, 80%, 85%, 90%, 95%, 97%, or 99% identity with that of a known sequence of an LMW-GS antigenic unit.

The “percent identity” of two amino acid sequences may be determined using the algorithm of Karlin and Altschul, Proc. Natl. Acad. Sci. USA 87:2264-68, 1990, modified as in Karlin and Altschul, Proc. Natl. Acad. Sci. USA 90:5873-77, 1993. Such an algorithm is incorporated into the NBLAST and XBLAST programs (version 2.0) of Altschul, et al. J. Mol. Biol. 215:403-10, 1990. BLAST protein searches can be performed with the XBLAST program to obtain amino acid sequences homologous to the protein molecules of interest. Where gaps exist between two sequences, Gapped BLAST can be utilized as described in Altschul et al., Nucleic Acids Res. 25(17):3389-3402, 1997. When utilizing BLAST and Gapped BLAST programs, the default parameters of the respective programs (e.g., XBLAST and NBLAST) can be used. Alternatively, PSI BLAST or PHI BLAST can be used to perform an iterated search which detects distant relationships between molecules. When utilizing BLAST, Gapped BLAST, PSI Blast and PHI Blast programs, the default parameters of the respective programs (e.g., of XBLAST and NBLAST) can be used (see, e.g., National Center for Biotechnology Information (NCBI) on the worldwide web). Another specific, non-limiting example of a mathematical algorithm utilized for the comparison of sequences is the algorithm of Myers and Miller, 1988, CABIOS 4:11 17. Such an algorithm is incorporated in the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. The percent identity between two sequences can be determined using techniques similar to those described above, with or without allowing gaps, such that any software for protein sequence alignment can be used. In calculating percent identity, typically only exact matches are counted.

In some embodiments, an antigenic unit comprises a CD-relevant epitope comprising a determinant that is recognized by lymphocytes. The CD-relevant epitope can be a peptide which is presented by a major histocompatibility complex (MHC) molecule and is capable of specifically binding to a T cell receptor. In certain embodiments, a CD-relevant epitope is a region of a T cell immunogen that is specifically bound by a T cell receptor. In certain embodiments, a CD epitope may include chemically active surface groupings of molecules such as amino acids, sugar side chains, phosphoryl, or sulfonyl groups. In certain embodiments, a CD epitope may have specific three-dimensional structural characteristics and/or specific charge characteristics.

The CD-relevant T cell epitope comprised within an antigenic unit may in some embodiments comprise a short peptide that is bound to a class II MHC molecule, thus forming a ternary complex that can be recognized by a T cell bearing a matching T cell receptor binding to the MHC/peptide complex with appropriate affinity. T cell epitopes that bind to MHC class II molecules are typically about 12-30 amino acids in length but can be longer. In the case of peptides that bind to MHC class II molecules, the same peptide and corresponding T cell epitope may share a common core segment but differ in the overall length due to flanking sequences of differing lengths upstream of the amino terminus of the epitope core sequence and downstream of its carboxy terminus, respectively. The T cell epitope may be classified as an immunogen if it elicits an immune response.

A skilled artisan would appreciate that an epitope can be formed both from contiguous amino acids or noncontiguous amino acids juxtaposed by tertiary folding of a protein. Epitopes formed from contiguous amino acids (linear epitopes) are typically retained on exposure to denaturing solvents, whereas epitopes formed by tertiary folding (conformational epitopes) are typically lost upon treatment with denaturing solvents. An epitope typically includes at least 3, 4, 5, 6, 7, 8, 9, 10, 11, 12, 13, 14 or 15 amino acids in a unique spatial conformation. Within a protein sequence, for example the amino acid sequences of a LMW-GS polypeptides, there are continuous epitopes that are linear sequences of amino acids that may be specifically bound by an antibody or T cell.

In certain embodiments, an LMW-GS may contain one or more wild-type (non-modified) epitopes having the amino acid sequences of one of SEQ ID NOs:1-9, and 21-48, 50-64.

In some embodiments, a de-epitoped LMW-GS as disclosed herein comprises at least 2 mutations within at least one antigenic unit or epitope, wherein the de-epitoped LMW-GS binds with less affinity to its relevant MHC protein than a wild-type counterpart, and/or wherein the de-epitoped LMW-GS activates T cells to a lesser extent than its wild-type counterpart, as further described herein below. In some embodiments, a de-epitoped LMW-GS as disclosed herein comprises at least 2 substitution mutations within at least one antigenic unit or epitope, wherein the de-epitoped LMW-GS binds with less affinity to its relevant MHC protein than a wild-type counterpart, and/or wherein the de-epitoped LMW-GS activates T cells to a lesser extent than its wild-type counterpart, as further described herein below.

Methods for making polypeptides comprising one or more mutations are well known to one of ordinary skills in the art. In some embodiments, the one or more mutations are conservative mutations. In some embodiments, the one or more mutations are non-conservative mutations. In some embodiments, the one or more mutations are a mixture of conservative and non-conservative mutations.

In some embodiments, the one or more mutations comprise a substitution, a deletion, or an insertion, or a combination thereof. In some embodiments, a mutation within an antigenic unit comprises a substitution. In some embodiments, a mutation within an antigenic unit comprises a deletion. In some embodiments, a mutation within an antigenic unit comprises an insertion. One skilled in the art would appreciate that one antigenic unit may comprise certain mutations, while another antigenic unit may comprise different mutations.

### Modified Epitope, X1, X2, X3, X4, Q, X6, X7, X8, X9

In some embodiments, the de-epitoped LMW-GS comprises at least one modified epitope comprising the amino acid sequence of X1, X2, X3, X4, Q, X6, X7, X8, X9.

In some embodiments, the amino acid at position X1 of at least one modified epitope is Pro, Ala, or Ser. In some embodiments, the amino acid at position X1 of 2, 3, 4, 5, 6, 7, 8, 9, 10, 11, 12, 13, 14, 15, 16, 17, 18, 19, 20, 21, 22, 23, 24, 25, 26, 27, 28, 29, or 30 modified epitopes is Pro, Ala, or Ser. In some embodiments, the amino acid at position X1 of at least 50%, 60%, 70%, 80%, 90% of the modified epitopes is Pro, Ala, or Ser.

In some embodiments, the amino acid at position X2 of at least one modified epitope is Phe, Ser, or Asp. In some embodiments, the amino acid at position X2 of 2, 3, 4, 5, 6, 7, 8, 9, 10, 11, 12, 13, 14, 15, 16, 17, 18, 19, 20, 21, 22, 23, 24, 25, 26, 27, 28, 29, or 30 modified epitopes is Phe, Ser, or Asp. In some embodiments, the amino acid at position X2 of at least 50%, 60%, 70%, 80%, 90% of the modified epitopes is Phe, Ser, or Asp.

In some embodiments, the amino acid at position X3 of at least one modified epitope is Pro or Ser. In some embodiments, the amino acid at position X3 of 2, 3, 4, 5, 6, 7, 8, 9, 10, 11, 12, 13, 14, 15, 16, 17, 18, 19, 20, 21, 22, 23, 24, 25, 26, 27, 28, 29, or 30 modified epitopes is Pro or Ser. In some embodiments, the amino acid at position X3 of at least 50%, 60%, 70%, 80%, 90% of the modified epitopes is Pro or Ser.

In some embodiments, the amino acid at position X4 of at least one modified epitope is Gln, Arg, Lys, His, or Glu. In some embodiments, the amino acid at position X4 of 2, 3, 4, 5, 6, 7, 8, 9, 10, 11, 12, 13, 14, 15, 16, 17, 18, 19, 20, 21, 22, 23, 24, 25, 26, 27, 28, 29, or 30 modified epitopes is Gln, Arg, Lys, His, or Glu. In some embodiments, the amino acid at position X4 of at least 50%, 60%, 70%, 80%, 90% of the modified epitopes is Gln, Arg, Lys, His, or Glu.

In some embodiments, the amino acid at position X6 of at least one modified epitope is Gln, Arg, Thr, His, or Glu. In some embodiments, the amino acid at position X6 of 2, 3, 4, 5, 6, 7, 8, 9, 10, 11, 12, 13, 14, 15, 16, 17, 18, 19, 20, 21, 22, 23, 24, 25, 26, 27, 28, 29, or 30 modified epitopes is Gln, Arg, Thr, His, or Glu. In some embodiments, the amino acid at position X6 of at least 50%, 60%, 70%, 80%, 90% of the modified epitopes is Gln, Arg, Thr, His, or Glu.

In some embodiments, the amino acid at position X7 of at least one modified epitope is Gln, Arg, Lys, His, Pro, or Glu. In some embodiments, the amino acid at position X7 of 2, 3, 4, 5, 6, 7, 8, 9, 10, 11, 12, 13, 14, 15, 16, 17, 18, 19, 20, 21, 22, 23, 24, 25, 26, 27, 28, 29, or 30 modified epitopes is Gln, Arg, Lys, His, Pro, or Glu. In some embodiments, the amino acid at position X7 of at least 50%, 60%, 70%, 80%, 90% of the modified epitopes is Gln, Arg, Lys, His, Pro, or Glu.

In some embodiments, the amino acid at position X8 of at least one modified epitope is Pro, Ser, or Gln. In some embodiments, the amino acid at position X8 of 2, 3, 4, 5, 6, 7, 8, 9, 10, 11, 12, 13, 14, 15, 16, 17, 18, 19, 20, 21, 22, 23, 24, 25, 26, 27, 28, 29, or 30 modified epitopes is Pro, Ser, or Gln. In some embodiments, the amino acid at position X8 of at least 50%, 60%, 70%, 80%, 90% of the modified epitopes is Pro, Ser, or Gln.

In some embodiments, the amino acid at position X9 of at least one modified epitope is Pro, Val, Gln, His, Phe, Ser, or Gly. In some embodiments, the amino acid at position X9 of 2, 3, 4, 5, 6, 7, 8, 9, 10, 11, 12, 13, 14, 15, 16, 17, 18, 19, 20, 21, 22, 23, 24, 25, 26, 27, 28, 29, or 30 modified epitopes is Pro, Val, Gln, His, Phe, Ser, or Gly. In some embodiments, the amino acid at position X9 of at least 50%, 60%, 70%, 80%, 90% of the modified epitopes is Pro, Val, Gln, His, Phe, Ser, or Gly.

### Modified Epitope, X1, X2, X3, Q, X5, X6, X7, X8, X9

In some embodiments, the de-epitoped LMW-GS comprises at least one modified epitope comprising the amino acid sequence of X1, X2, X3, Q, X5, X6, X7, X8, X9.

In some embodiments, the amino acid at position X1 of at least one modified epitope is Phe, Ser, or Asp. In some embodiments, the amino acid at position X1 of 2, 3, 4, 5, 6, 7, 8, 9, 10, 11, 12, 13, 14, 15, 16, 17, 18, 19, 20, 21, 22, 23, 24, 25, 26, 27, 28, 29, or 30 modified epitopes is Phe, Ser, or Asp. In some embodiments, the amino acid at position X1 of at least 50%, 60%, 70%, 80%, 90% of the modified epitopes is Phe, Ser, or Asp.

In some embodiments, the amino acid at position X2 of at least one modified epitope is Pro or Ser. In some embodiments, the amino acid at position X2 of 2, 3, 4, 5, 6, 7, 8, 9, 10, 11, 12, 13, 14, 15, 16, 17, 18, 19, 20, 21, 22, 23, 24, 25, 26, 27, 28, 29, or 30 modified epitopes is Pro or Ser. In some embodiments, the amino acid at position X2 of at least 50%, 60%, 70%, 80%, 90% of the modified epitopes is Pro or Ser.

In some embodiments, the amino acid at position X3 of at least one modified epitope is Gln, Arg, Lys, His, or Glu. In some embodiments, the amino acid at position X3 of 2, 3, 4, 5, 6, 7, 8, 9, 10, 11, 12, 13, 14, 15, 16, 17, 18, 19, 20, 21, 22, 23, 24, 25, 26, 27, 28, 29, or 30 modified epitopes is Gln, Arg, Lys, His, or Glu. In some embodiments, the amino acid at position X3 of at least 50%, 60%, 70%, 80%, 90% of the modified epitopes is Gln, Arg, Lys, His, or Glu.

In some embodiments, the amino acid at position X5 of at least one modified epitope is Gln, Arg, Thr, His, or Glu. In some embodiments, the amino acid at position X5 of 2, 3, 4, 5, 6, 7, 8, 9, 10, 11, 12, 13, 14, 15, 16, 17, 18, 19, 20, 21, 22, 23, 24, 25, 26, 27, 28, 29, or 30 modified epitopes is Gln, Arg, Thr, His, or Glu. In some embodiments, the amino acid at position X5 of at least 50%, 60%, 70%, 80%, 90% of the modified epitopes is Gln, Arg, Thr, His, or Glu.

In some embodiments, the amino acid at position X6 of at least one modified epitope is Gln, Arg, Lys, His, Pro, or Glu. In some embodiments, the amino acid at position X6 of 2, 3, 4, 5, 6, 7, 8, 9, 10, 11, 12, 13, 14, 15, 16, 17, 18, 19, 20, 21, 22, 23, 24, 25, 26, 27, 28, 29, or 30 modified epitopes is Gln, Arg, Lys, His, Pro, or Glu. In some embodiments, the amino acid at position X6 of at least 50%, 60%, 70%, 80%, 90% of the modified epitopes is Gln, Arg, Lys, His, Pro, or Glu.

In some embodiments, the amino acid at position X7 of at least one modified epitope is Pro, Ser, or Gln. In some embodiments, the amino acid at position X7 of 2, 3, 4, 5, 6, 7, 8, 9, 10, 11, 12, 13, 14, 15, 16, 17, 18, 19, 20, 21, 22, 23, 24, 25, 26, 27, 28, 29, or 30 modified epitopes is Pro, Ser, or Gln. In some embodiments, the amino acid at position X7 of at least 50%, 60%, 70%, 80%, 90% of the modified epitopes is Pro, Ser, or Gln.

In some embodiments, the amino acid at position X8 of at least one modified epitope is Pro, Val, Gln, His, Phe, Ser, or Gly. In some embodiments, the amino acid at position X8 of 2, 3, 4, 5, 6, 7, 8, 9, 10, 11, 12, 13, 14, 15, 16, 17, 18, 19, 20, 21, 22, 23, 24, 25, 26, 27, 28, 29, or 30 modified epitopes is Pro, Val, Gln, His, Phe, Ser, or Gly. In some embodiments, the amino acid at position X8 of at least 50%, 60%, 70%, 80%, 90% of the modified epitopes is Pro, Val, Gln, His, Phe, Ser, or Gly.

In some embodiments, the amino acid at position X9 of at least one modified epitope is Phe, Pro, Gln, Ser, Gly, or Ile. In some embodiments, the amino acid at position X9 of 2, 3, 4, 5, 6, 7, 8, 9, 10, 11, 12, 13, 14, 15, 16, 17, 18, 19, 20, 21, 22, 23, 24, 25, 26, 27, 28, 29, or 30 modified epitopes is Phe, Pro, Gln, Ser, Gly, or Ile. In some embodiments, the amino acid at position X9 of at least 50%, 60%, 70%, 80%, 90% of the modified epitopes is Phe, Pro, Gln, Ser, Gly, or Ile.

### Nucleotides, Vectors, and Host Cells

The present disclosure also encompasses isolated polynucleotides, which encode any of the above described de-epitoped LMW-GS. Such polynucleotides may be used to express the above described de-epitoped LMW-GS in host cells (e.g., bacteria, plants, yeast, or mammalian cell hosts).

In some embodiments, polynucleotides encoding de-epitoped LMW-GS are expressed in a plant cell, a mammalian cell, or a microorganism. In some embodiments, polynucleotides encoding de-epitoped LMS-GS are comprised within an expression vector, wherein the expression vector is used to express the de-epitoped LMW-GS in a plant cell, a mammalian cell, or a microorganism. In some embodiments, a microorganism comprises bacteria, archaea, fungi (yeasts and molds), and algae. In some embodiments a fungus comprises Aspergillus.

The term “nucleotide”, “nucleotide sequence”, “polynucleotide”, “polynucleotide sequence”, or “nucleic acid molecule” or the like, as used herein may encompass DNA molecules, RNA molecules or modified RNA molecules. A nucleic acid molecule may be single-stranded or double-stranded. In some embodiments, a nucleotide comprises a modified nucleotide. In some embodiments, a nucleotide comprises an mRNA. In some embodiments, a nucleotide comprises a modified mRNA. In some embodiments, a nucleotide comprises a modified mRNA, wherein the modified mRNA comprises a 5′-capped mRNA. In some embodiments, a modified mRNA comprises a molecule in which some of the nucleosides have been replaced by either naturally modified or synthetic nucleosides. In some embodiments, a modified nucleotide comprises a modified mRNA comprising a 5′-capped mRNA and wherein some of the nucleosides have been replaced by either naturally modified or synthetic nucleosides.

A skilled artisan would appreciate that the terms “polynucleotide”, “nucleic acid sequence”, “nucleic acid”, and variations thereof may be generic encompassing polydeoxyribonucleotides (containing 2-deoxy-D-ribose), to polyribonucleotides (containing D-ribose), to any other type of polynucleotide that is an N-glycoside of a purine or pyrimidine base, and to other polymers containing non-nucleotide backbones, provided that the polymers contain nucleobases in a configuration that allows for base pairing and base stacking, as found in DNA and RNA. Thus, these terms include known types of nucleic acid sequence modifications, for example, substitution of one or more of the naturally occurring nucleotides with an analog, and inter-nucleotide modifications.

Methods of introducing nucleic acid alterations to a gene of interest are well known in the art [see for example Menke D. Genesis (2013) 51:-618; Capecchi, Science (1989) 244:1288-1292; Santiago et al. Proc Natl Acad Sci USA (2008) 105:5809-5814; International Patent Application Nos. WO 2014085593, WO 2009071334 and WO 2011146121; U.S. Pat. Nos. 8,771,945, 8,586,526, 6,774,279 and UP Patent Application Publication Nos. 20030232410, 20050026157, US20060014264; the contents of which are incorporated by reference in their entireties] and include targeted homologous recombination, site specific recombinases, PB transposases and genome editing by engineered nucleases. Agents for introducing nucleic acid alterations to a gene of interest can be designed by publicly available sources or obtained commercially. In some embodiments, the generation of the alterations in the sequences of the genes may be achieved by screening sequences of existing plants in search of an existing variant of the desired sequence. Then, this existing sequence can be introduced into the genome of the target genome by crossbreeding, or by gene editing. In other embodiments the desired variations can be introduced by introducing random mutagenesis, followed by screening for variants where the desired mutations occurred, followed by crossbreeding.

Methods for qualifying efficacy and detecting sequence alteration are well known in the art and include, but not limited to, DNA sequencing, electrophoresis, an enzyme-based mismatch detection assay and a hybridization assay such as PCR, RT-PCR, RNase protection, in-situ hybridization, primer extension, Southern blot, Northern Blot and dot blot analysis.

In some embodiments, disclosed herein is an expression vector comprising the isolated polynucleotide encoding any of the de-epitoped LMW-GS disclosed herein, operatively linked to a transcriptional regulatory sequence so as to allow expression of the de-epitoped LMW-GS in a plant cell. In some embodiments, disclosed herein are host cells comprising the expression vector comprising the isolated polynucleotide encoding any of the de-epitoped LMW-GS disclosed herein. In some embodiments, the host cell comprises a plant cell. In some embodiments, a plant cell comprises a wheat cell. In some embodiments, a plant cell comprises a corn cell. In some embodiments, a plant cell comprises a tobacco cell. In some embodiments, the host cell comprises a yeast cell. In some embodiments, the host cell comprises a bacterial cell. In some embodiments, the host cell comprises a mammalian cell.

In some embodiments, a plant cell comprises the de-epitoped LMW-GS disclosed herein. In some embodiments, a plant cell comprises the de-epitoped LMW-GS disclosed herein, wherein the de-epitoped LMW-GS is from the same species of plant compared with the plant cell. In some embodiments, a plant cell comprises a de-epitoped LMW-GS disclosed herein, wherein the de-epitoped LMW-GS is from a heterologous species of plant compared with the plant cell.

In some embodiments, a plant cell comprises the de-epitoped LMW-GS disclosed herein. In some embodiments, a bacterial cell comprises the de-epitoped LMW-GS disclosed herein. In some embodiments, a yeast cell comprises the de-epitoped LMW-GS disclosed herein. In some embodiments, a mammalian cell comprises the de-epitoped LMW-GS disclosed herein.

As used herein, the term “vector” refers to discrete elements that are used to introduce heterologous nucleic acids into cells for either expression or replication thereof. An expression vector includes vectors capable of expressing nucleic acids that are operatively linked with regulatory sequences, such as promoter regions, which are capable of affecting expression of such nucleic acids. Thus, an expression vector may refer to a DNA or RNA construct, such as a plasmid, a phage, recombinant virus or other vector that, upon introduction into an appropriate host cell, results in expression of the nucleic acids. Appropriate expression vectors are well known to those of skill in the art and include those that are replicable in prokaryotic cells and/or eukaryotic cells, and those that remain episomal or those which integrate into the host cell genome.

The term “recombinant host cell” (or simply “host cell”) as used herein refers to a cell into which a recombinant expression vector has been introduced. It should be understood that such terms are intended to refer not only to the particular subject cell but to the progeny of such a cell. Because certain modifications may occur in succeeding generations due to either mutation or environmental influences, such progeny may not, in fact, be identical to the parent cell, but are still included within the scope of the term “host cell” as used herein.

Commonly used expression systems for heterologous protein production include bacterial cells (e.g. E. coli), fungal cells (e.g. S. cerevisiae cells), plant cells (e.g. wheat, tobacco, maize), insect cells (lepidopteran cells), and mammalian cells (for example but not limited to Chinese Hamster Ovary cells).

Various methods can be used to introduce the expression vector of some embodiments of the present disclosure into cells. Such methods are generally described in Sambrook et al., Molecular Cloning: A Laboratory Manual, Cold Springs Harbor Laboratory, New York (1989, 1992), in Ausubel et al., Current Protocols in Molecular Biology, John Wiley and Sons, Baltimore, Md. (1989), Chang et al., Somatic Gene Therapy, CRC Press, Ann Arbor, Mich. (1995), Vega et al., Gene Targeting, CRC Press, Ann Arbor Mich. (1995), Vectors: A Survey of Molecular Cloning Vectors and Their Uses, Butterworths, Boston Mass. (1988) and Gilboa et at. [Biotechniques 4 (6): 504-512, 1986] and include, for example, stable or transient transfection, lipofection, electroporation and infection with recombinant viral vectors. In addition, see U.S. Pat. Nos. 5,464,764 and 5,487,992 for positive-negative selection methods.

As used herein, the term “promoter” refers to a region of DNA which lies upstream of the transcriptional initiation site of a gene to which RNA polymerase binds to initiate transcription of RNA. The promoter controls where (e.g., which portion of a plant, which organ within an animal, etc.) and/or when (e.g., which stage or condition in the lifetime of an organism) the gene is expressed.

Any suitable promoter sequence can be used by the nucleic acid construct encoding the de-epitoped LMW-GS. In some embodiments, the promoter is a constitutive promoter, a tissue-specific promoter or a plant-specific promoter, for example but not limited to a wheat promoter.

Suitable wheat specific promoters include, but are not limited to those described in Smirnova, O. G. and Kochetov, A. V. Russ J Genet Appl Res (2012) 2: 434. www(dot)doi(dot)org/10(dot)1134/S2079059712060123.

The nucleic acid construct disclosed herein can be utilized to stably or transiently transform plant cells. In stable transformation, the exogenous polynucleotide disclosed herein is integrated into the plant genome and as such it represents a stable and inherited trait. In transient transformation, the exogenous polynucleotide is expressed by the cell transformed but it is not integrated into the genome and as such it represents a transient trait.

There are various methods of introducing foreign genes into both monocotyledonous and dicotyledonous plants (e.g. Potrykus, I., Annu. Rev. Plant. Physiol., Plant. Mol. Biol. (1991) 42:205-225; Shimamoto et al., Nature (1989) 338:274-276).

Viruses that have been shown to be useful for the transformation of plant hosts include CaMV, TMV and BV. Transformation of plants using plant viruses is described in U.S. Pat. No. 4,855,237 (BGV), EP-A 67,553 (TMV), Japanese Published Application No. 63-14693 (TMV), EPA 194,809 (BV), EPA 278,667 (BV); and Gluzman, Y. et al., Communications in Molecular Biology: Viral Vectors, Cold Spring Harbor Laboratory, New York, pp. 172-189 (1988). Pseudovirus particles for use in expressing foreign DNA in many hosts, including plants, is described in WO 87/06261.

Techniques for inoculation of viruses to plants may be found in Foster and Taylor, eds. “Plant Virology Protocols: From Virus Isolation to Transgenic Resistance (Methods in Molecular Biology (Humana Pr), Vol 81)”, Humana Press, 1998; Maramorosh and Koprowski, eds. “Methods in Virology” 7 vols, Academic Press, New York 1967-1984; Hill, S. A. “Methods in Plant Virology”, Blackwell, Oxford, 1984; Walkey, D. G. A. “Applied Plant Virology”, Wiley, New York, 1985; and Kado and Agrawa, eds. “Principles and Techniques in Plant Virology”, Van Nostrand-Reinhold, New York.

In one embodiment, the plant host cell in which the expression construct is transfected does not naturally express gluten polypeptides (i.e., derived from a non-gluten plant). Thus, in one embodiment, the host cell can be amaranth, buckwheat, rice (brown, white, wild), corn millet, quinoa, sorghum, Montina. Job's tears and teff.

In another embodiment, the plant host cell in which the expression construct is transfected expresses wild-type gluten polypeptides. Such host cells include but are not limited to wheat varieties such as spelt, kamut, farro and durum, bulger, semolina, barley, rye, triticale, Triticum (wheat cultivars—fielder, spelling, bobwhite, Cheyenne, chinse spring and Mjolnir) and oats. It will be appreciated that in host cells that naturally express gluten polypeptides, it is expected to have down-regulated expression of the wild-type gluten polypeptides. Methods of down-regulating expression of wild-type gluten polypeptides are known in the art and include for example the use of RNA silencing agent and DNA editing agents. Examples of RNA silencing agents include, but are not limited to siRNA, miRNA, antisense molecules, DNAzyme, RNAzyme. One method of downregulating expression of gluten polypeptides has been described in Sinchez-Le6n, Susana et al. “Low-gluten, Nontransgenic Wheat Engineered with CRISPR/Cas9.” Plant Biotechnology Journal 16.4 (2018): 902-910. PMC, the contents of which are incorporated herein by reference.

### Methods of Producing De-Epitoped Low Molecular Weight Glutenin Subunits (LMW-GS)

Disclosed herein is a method of producing a de-epitoped LMW-GS comprising (a) culturing cells that comprise an expression vector comprising a polynucleotide encoding any of the de-epitoped LMW-GS disclosed herein, wherein the culturing is under conditions allowing for expression of the de-epitoped LMW-GS in the cells; and (b) collecting the expressed de-epitoped LMW-GS. In some embodiments, the cell comprises a plant cell. In some embodiments, the cell comprises a wheat cell. In some embodiments, the cell comprises a mammalian cell. In some embodiments, the cell comprises a yeast cell. In some embodiments, the cell comprises a microorganism.

In some embodiments, a method of producing a de-epitoped LMW-GS comprises use of a cell free in-vitro translation system, as is well known in the art for example but not limited to methods reviewed in Dondapati et al. (2020) BioDrugs 34(3):327-348, where the method comprises (a) translating the de-epitoped LMW-GS in a cell free translation system and (b) collecting the translated de-epitoped LMW-GS

In some embodiments, a method of producing a de-epitoped LMW-GS produces a de-epitoped LMW-GS comprising substitution mutations, or deletion mutations, or a combination thereof in the repeating antigenic unit of the LMW glutenin.

In some embodiments, a method of producing a de-epitoped LMW-GS produces a de-epitoped LMW-GS comprising substitution mutations in at least 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 repeating antigenic units of the de-epitoped LMW-GS.

In some embodiments, a method of producing a de-epitoped LMW-GS produces a de-epitoped LMW-GS comprising substitution mutations in at least 50%, 60%, 70%, 80%, 90% or all of the antigenic units of the de-epitoped LMW-GS.

Recovery of the recombinant polypeptide is affected following an appropriate time in culture. The phrase “recovering the recombinant polypeptide” encompasses collecting the whole culture medium, for example a fermentation medium containing the modified LMW-GS. In some embodiments, collecting comprises additional steps of separation or purification. In some embodiments, collecting does not comprise additional steps of separation or purification.

Notwithstanding the above, modified LMW-GS disclosed herein may be purified using a variety of standard protein purification techniques, such as, but not limited to, affinity chromatography, ion exchange chromatography, filtration, electrophoresis, hydrophobic interaction chromatography, gel filtration chromatography, reverse phase chromatography, concanavalin A chromatography, chromatofocusing and differential solubilization.

### Method of De-Epitoping Low Molecular Weight Glutenin Subunits (LMW-GS)

In some embodiments, disclosed herein is a method of de-epitoping LMW-GS, comprising changing one or more amino acid residues of at least one epitope of a wild-type LMW-GS, the epitope comprises the amino acid sequence of X1, X2, X3, X4, Q, X6, X7, X8, X9, wherein X1 is changed to Pro, Ala, or Ser, X2 is changed to Phe, Ser, or Asp, X3 is changed to Ser or Pro, X4 is changed to Gln, Arg, Lys, His, or Glu, X6 is changed to Gln, Arg, Thr, His, or Glu, X7 is changed to Gln, Arg, Lys, His, Pro, or Glu, X8 is changed to Pro, Ser, or Gln, and X9 is changed to Pro, Val, Gln, His, Phe, Ser, or Gly, thereby generating a de-epitoped LMW-GS with one or more modified epitopes.

In some embodiments, the amino acid at position X1 of at least one epitope of a wild-type LMW-GS is changed to Pro, Ala, or Ser. In some embodiments, the amino acid at position X1 of 2, 3, 4, 5, 6, 7, 8, 9, 10, 11, 12, 13, 14, 15, 16, 17, 18, 19, 20, 21, 22, 23, 24, 25, 26, 27, 28, 29, or 30 epitopes of a wild-type LMW-GS is changed to Pro, Ala, or Ser. In some embodiments, the amino acid at position X1 of at least 50%, 60%, 70%, 80%, 90%, 95% of the epitopes of a wild-type LMW-GS is changed to Pro, Ala, or Ser.

In some embodiments, the amino acid at position X2 of at least one epitope of a wild-type LMW-GS is changed to Phe, Ser, or Asp. In some embodiments, the amino acid at position X2 of 2, 3, 4, 5, 6, 7, 8, 9, 10, 11, 12, 13, 14, 15, 16, 17, 18, 19, 20, 21, 22, 23, 24, 25, 26, 27, 28, 29, or 30 epitopes of a wild-type LMW-GS is changed to Phe, Ser, or Asp. In some embodiments, the amino acid at position X2 of at least 50%, 60%, 70%, 80%, 90%, 95% of the epitopes of a wild-type LMW-GS is changed to Phe, Ser, or Asp.

In some embodiments, the amino acid at position X3 of at least one epitope of a wild-type LMW-GS is changed to Pro or Ser. In some embodiments, the amino acid at position X3 of 2, 3, 4, 5, 6, 7, 8, 9, 10, 11, 12, 13, 14, 15, 16, 17, 18, 19, 20, 21, 22, 23, 24, 25, 26, 27, 28, 29, or 30 epitopes of a wild-type LMW-GS is changed to Pro or Ser. In some embodiments, the amino acid at position X3 of at least 50%, 60%, 70%, 80%, 90% of the epitopes of a wild-type LMW-GS is changed to Pro or Ser.

In some embodiments, the amino acid at position X4 of at least one epitope of a wild-type LMW-GS is changed to Gln, Arg, Lys, His, or Glu. In some embodiments, the amino acid at position X4 of 2, 3, 4, 5, 6, 7, 8, 9, 10, 11, 12, 13, 14, 15, 16, 17, 18, 19, 20, 21, 22, 23, 24, 25, 26, 27, 28, 29, or 30 epitopes of a wild-type LMW-GS is changed to Gln, Arg, Lys, His, or Glu. In some embodiments, the amino acid at position X4 of at least 50%, 60%, 70%, 80%, 90% of the epitopes of a wild-type LMW-GS is changed to Gln, Arg, Lys, His, or Glu.

In some embodiments, the amino acid at position X6 of at least one epitope of a wild-type LMW-GS is changed to Gln, Arg, Thr, His, or Glu. In some embodiments, the amino acid at position X6 of 2, 3, 4, 5, 6, 7, 8, 9, 10, 11, 12, 13, 14, 15, 16, 17, 18, 19, 20, 21, 22, 23, 24, 25, 26, 27, 28, 29, or 30 epitopes of a wild-type LMW-GS is changed to Gln, Arg, Thr, His, or Glu. In some embodiments, the amino acid at position X6 of at least 50%, 60%, 70%, 80%, 90%, 95% of the epitopes of a wild-type LMW-GS is changed to Gln, Arg, Thr, His, or Glu.

In some embodiments, the amino acid at position X7 of at least one epitope of a wild-type LMW-GS is changed to Gln, Arg, Lys, His, Pro, or Glu. In some embodiments, the amino acid at position X7 of 2, 3, 4, 5, 6, 7, 8, 9, 10, 11, 12, 13, 14, 15, 16, 17, 18, 19, 20, 21, 22, 23, 24, 25, 26, 27, 28, 29, or 30 epitopes of a wild-type LMW-GS is changed to Gln, Arg, Lys, His, Pro, or Glu. In some embodiments, the amino acid at position X7 of at least 50%, 60%, 70%, 80%, 90%, 95% of the epitopes of a wild-type LMW-GS is changed to Gln, Arg, Lys, His, Pro, or Glu.

In some embodiments, the amino acid at position X8 of at least one epitope of a wild-type LMW-GS is changed to Pro, Ser, or Gln. In some embodiments, the amino acid at position X8 of 2, 3, 4, 5, 6, 7, 8, 9, 10, 11, 12, 13, 14, 15, 16, 17, 18, 19, 20, 21, 22, 23, 24, 25, 26, 27, 28, 29, or 30 epitopes of a wild-type LMW-GS is changed to Pro, Ser, or Gln. In some embodiments, the amino acid at position X8 of at least 50%, 60%, 70%, 80%, 90%, 95% of the epitopes of a wild-type LMW-GS is changed to Pro, Ser, or Gln.

In some embodiments, the amino acid at position X9 of at least one epitope of a wild-type LMW-GS is changed to Pro, Val, Gln, His, Phe, Ser, or Gly. In some embodiments, the amino acid at position X9 of 2, 3, 4, 5, 6, 7, 8, 9, 10, 11, 12, 13, 14, 15, 16, 17, 18, 19, 20, 21, 22, 23, 24, 25, 26, 27, 28, 29, or 30 epitopes of a wild-type LMW-GS is changed to Pro, Val, Gln, His, Phe, Ser, or Gly. In some embodiments, the amino acid at position X9 of at least 50%, 60%, 70%, 80%, 90%, 95% of the epitopes of a wild-type LMW-GS is changed to Pro, Val, Gln, His, Phe, Ser, or Gly.

In another aspect, disclosed herein is a method of de-epitoping LMW-GS, comprising changing one or more amino acid residues of at least one epitope of a wild-type LMW-GS, the epitope comprises the amino acid sequence of X1, X2, X3, Q, X5, X6, X7, X8, X9, wherein X1 is changed to Phe, Ser, or Asp, X2 is changed to Ser or Pro, X3 is changed to Gln, Arg, Lys, His, or Glu, X5 is changed to Gln, Arg, Thr, His, or Glu, X6 is changed to Gln, Arg, Lys, His, Pro, or Glu, X7 is changed to Pro, Ser, or Gln, X8 is changed to Pro, Val, Gln, His, Phe, Ser, or Gly, and X9 is changed to Phe, Pro, Gln, Ser, Gly, or Ile, thereby generating a de-epitoped LMW-GS with one or more modified epitopes.

In some embodiments, the amino acid at position X1 of at least one epitope of a wild-type LMW-GS is changed to Phe, Ser, or Asp. In some embodiments, the amino acid at position X1 of 2, 3, 4, 5, 6, 7, 8, 9, 10, 11, 12, 13, 14, 15, 16, 17, 18, 19, 20, 21, 22, 23, 24, 25, 26, 27, 28, 29, or 30 epitopes of a wild-type LMW-GS is changed to Phe, Ser, or Asp. In some embodiments, the amino acid at position X1 of at least 50%, 60%, 70%, 80%, 90%, 95% of the epitopes of a wild-type LMW-GS is changed to Phe, Ser, or Asp.

In some embodiments, the amino acid at position X2 of at least one epitope of a wild-type LMW-GS is changed to Ser or Pro. In some embodiments, the amino acid at position X2 of 2, 3, 4, 5, 6, 7, 8, 9, 10, 11, 12, 13, 14, 15, 16, 17, 18, 19, 20, 21, 22, 23, 24, 25, 26, 27, 28, 29, or 30 epitopes of a wild-type LMW-GS is changed to Ser or Pro. In some embodiments, the amino acid at position X2 of at least 50%, 60%, 70%, 80%, 90%, 95% of the epitopes of a wild-type LMW-GS is changed to Ser or Pro.

In some embodiments, the amino acid at position X3 of at least one epitope of a wild-type LMW-GS is changed to Gln, Arg, Lys, His, or Glu. In some embodiments, the amino acid at position X3 of 2, 3, 4, 5, 6, 7, 8, 9, 10, 11, 12, 13, 14, 15, 16, 17, 18, 19, 20, 21, 22, 23, 24, 25, 26, 27, 28, 29, or 30 epitopes of a wild-type LMW-GS is changed to Gln, Arg, Lys, His, or Glu. In some embodiments, the amino acid at position X3 of at least 50%, 60%, 70%, 80%, 90%, 95% of the epitopes of a wild-type LMW-GS is changed to Gln, Arg, Lys, His, or Glu.

In some embodiments, the amino acid at position X5 of at least one epitope of a wild-type LMW-GS is changed to Gln, Arg, Thr, His, or Glu. In some embodiments, the amino acid at position X5 of 2, 3, 4, 5, 6, 7, 8, 9, 10, 11, 12, 13, 14, 15, 16, 17, 18, 19, 20, 21, 22, 23, 24, 25, 26, 27, 28, 29, or 30 epitopes of a wild-type LMW-GS is changed to Gln, Arg, Thr, His, or Glu. In some embodiments, the amino acid at position X5 of at least 50%, 60%, 70%, 80%, 90%, 95% of the epitopes of a wild-type LMW-GS is changed to Gln, Arg, Thr, His, or Glu.

In some embodiments, the amino acid at position X6 of at least one epitope of a wild-type LMW-GS is changed to Gln, Arg, Lys, His, Pro, or Glu. In some embodiments, the amino acid at position X6 of 2, 3, 4, 5, 6, 7, 8, 9, 10, 11, 12, 13, 14, 15, 16, 17, 18, 19, 20, 21, 22, 23, 24, 25, 26, 27, 28, 29, or 30 epitopes of a wild-type LMW-GS is changed to Gln, Arg, Lys, His, Pro, or Glu. In some embodiments, the amino acid at position X6 of at least 50%, 60%, 70%, 80%, 90%, 95% of the epitopes of a wild-type LMW-GS is changed to Gln, Arg, Lys, His, Pro, or Glu.

In some embodiments, the amino acid at position X7 of at least one epitope of a wild-type LMW-GS is changed to Pro, Ser, or Gln. In some embodiments, the amino acid at position X7 of 2, 3, 4, 5, 6, 7, 8, 9, 10, 11, 12, 13, 14, 15, 16, 17, 18, 19, 20, 21, 22, 23, 24, 25, 26, 27, 28, 29, or 30 epitopes of a wild-type LMW-GS is changed to Pro, Ser, or Gln. In some embodiments, the amino acid at position X7 of at least 50%, 60%, 70%, 80%, 90%, 95% of the epitopes of a wild-type LMW-GS is changed to Pro, Ser, or Gln.

In some embodiments, the amino acid at position X8 of at least one epitope of a wild-type LMW-GS is changed to Pro, Val, Gln, His, Phe, Ser, or Gly. In some embodiments, the amino acid at position X8 of 2, 3, 4, 5, 6, 7, 8, 9, 10, 11, 12, 13, 14, 15, 16, 17, 18, 19, 20, 21, 22, 23, 24, 25, 26, 27, 28, 29, or 30 epitopes of a wild-type LMW-GS is changed to Pro, Val, Gln, His, Phe, Ser, or Gly. In some embodiments, the amino acid at position X8 of at least 50%, 60%, 70%, 80%, 90%, 95% of the epitopes of a wild-type LMW-GS is changed to Pro, Val, Gln, His, Phe, Ser, or Gly.

In some embodiments, the amino acid at position X9 of at least one epitope of a wild-type LMW-GS is changed to Phe, Pro, Gln, Ser, Gly, or Ile. In some embodiments, the amino acid at position X9 of 2, 3, 4, 5, 6, 7, 8, 9, 10, 11, 12, 13, 14, 15, 16, 17, 18, 19, 20, 21, 22, 23, 24, 25, 26, 27, 28, 29, or 30 epitopes of a wild-type LMW-GS is changed to Phe, Pro, Gln, Ser, Gly, or Ile. In some embodiments, the amino acid at position X9 of at least 50%, 60%, 70%, 80%, 90%, 95% of the epitopes of a wild-type LMW-GS is changed to Phe, Pro, Gln, Ser, Gly, or Ile.

In some embodiments, the mutations or changes to the above epitopes do not disrupt the function of the LMW-GS (for example but not limited to not disrupting the function of the modified LMW-GS relative to the function of the corresponding un-modified LMW-GS).

In some embodiments, the mutations or changes to the above epitopes do not disrupt at least one of the following characteristics: (1) the dough strengthening ability of the LMW-GS; (2) the dough elasticity promoting ability of the LMW-GS; (3) the dough rising promoting ability of the LMW-GS; (4) the growth of a plant comprising the modified LMW-GS, for example but not limited to wheat, wherein the production of seeds, number of seeds, or size of seeds is not disrupted; (5) native protein-protein interactions of the de-epitoped LMW-GS (e.g., the modified LMW-GS retains the ability to form substantially the same protein-protein interactions as the corresponding un-mutated LMW-GS); (6) the three-dimensional structure of the LMW-GS (e.g., the de-epitoped LMW-GS retains substantially the same three-dimensional structure as the corresponding un-modified LMW-GS); (7) the folding of the LMW-GS (e.g., the de-epitoped LMW-GS retains substantially the same protein folding as the corresponding un-modified LMW-GS); (8) the translation of the LMW-GS (e.g., the de-epitoped LMW-GS is translated with the same timing, at the same rate, to the same levels, etc. as the corresponding un-modified LMW-GS); (9) the normal cellular localization of the LMW-GS (e.g., the de-epitoped LMW-GS retains substantially the same cellular localization as the corresponding un-modified LMW-GS); and (10) any post-translational modifications on the LMW-GS (e.g., the de-epitoped LMW-GS retains substantially the same post-translational modification profile as the corresponding un-modified LMW-GS).

In some embodiments, the method of de-epitoping LMW-GS disclosed herein produces a modified LMW-GS that binds with a poorer affinity to celiac related MHC class II proteins (e.g. HLA-DQ2.5, HLA-DQ8, HLA-DQ2.2, or HLA-DQ7.5) or to T cells derived from a celiac patient as compared to a corresponding non-modified LMW-GS.

In some embodiments, the method of de-epitoping LMW-GS disclosed herein produces a modified LMW-GS that has abrogated binding to celiac related MHC class II proteins (e.g., HLA-DQ2.5, HLA-DQ8, HLA-DQ2.2, or HLA-DQ7.5) or to T cells derived from a celiac patient as compared to a corresponding non-modified LMW-GS. Methods of measuring the binding of LMW-GS, or epitopes of the LMW-GS, to Celiac related MHC class II proteins or to T cells are well-known in the art.

In some embodiments, the method of de-epitoping LMW-GS disclosed herein produces a modified LMW-GS that activates T cells derived from a celiac patient to a lesser extent, e.g. by at least 10%, 20%, 30%, 40%, 50%, 60%, 70%, 80%, 90% or 100% less than a corresponding non-mutated LMW-GS. Methods and assays for determining T cell activation are well-known in the art, e.g. measurement of IFN-γ secretion using ELISA.

In some embodiments, the method of de-epitoping LMW-GS disclosed herein further comprises one or more of the following: (1) analyzing binding affinity of the de-epitoped LMW-GS to one or more of MHC Class II molecules to identify a de-epitoped LMW-GS with reduced binding affinity to the MHC Class II molecules (e.g. HLA-DQ2.5, HLA-DQ8, HLA-DQ2.2, or HLA-DQ7.5); or (2) analyzing immunogenicity of the de-epitoped LMW-GS to identify a de-epitoped LMW-GS with reduced immunogenicity. Methods and assays for determining immunogenicity are well-known in the art. In some embodiments, immunogenicity can be determined by analyzing the binding of the de-epitoped LMW-GS to T cells derived from a celiac patient or analyzing the activation of T cells by the de-epitoped LMW-GS.

### Food Products

Disclosed herein are uses of the modified de-epitoped LMW-GS for producing food and beverage products that may be particularly beneficial for subjects suffering from a gluten sensitivity, including a glutenin sensitivity, for example a subject suffering from CD or any related irritation of the intestine or bowels. These food products may also benefit a subject suffering from non-celiac gluten sensitivity. In some embodiments, the de-epitoped LMW-GS described herein is for the preparation of foods and or beverages suitable for consumption by a subject having celiac disease. In some embodiments, the de-epitoped LMW-GS disclosed herein may be used in the preparation of meat products, cheese, and vegetarian alternatives to meat products.

In certain embodiments, the de-epitoped LMW-GS disclosed herein can be used in the preparation of edible flour. In some embodiments, disclosed herein is a flour comprising any of the de-epitoped LMW-GS described herein. In some embodiments, such flour does not contain any CD-relevant T cell epitope, or one or more of the CD-relevant T cell epitopes in such flour are mutated. In some embodiments, a flour comprising any of the de-epitoped LMW-GS disclosed herein is derived from a plant having reduced or no wild type LMW-GS. In some embodiments, a flour comprising any of the de-epitoped LMW-GS disclosed herein is derived from a plant lacking other gluten protein components. In some embodiments, a flour comprising any of the de-epitoped LMW-GS disclosed herein is derived from a plant expressing a reduced percentage of gluten proteins. In some embodiments, a plant from which a flour comprising a de-epitoped LMW-GS may have about 75% reduction of gluten proteins. Examples of plants (e.g., grains) from which a flour comprising a de-epitoped LMW-GS disclosed herein are derived include, but are not limited to, amaranth, wheat, buckwheat, rice (brown, white, wild), corn millet, quinoa, sorghum, and teff.

In some embodiments, disclosed herein is a modified wheat expressing a de-epitoped LMW-GS disclosed herein. In some embodiments, the modified wheat has reduced or no wild type LMW-GS. In some embodiments, disclosed herein is a flour comprising any of the de-epitoped LMW-GS disclosed herein, wherein the flour is derived from the modified wheat described herein.

In some embodiments, disclosed herein is a modified corn expressing a de-epitoped LMW-GS disclosed herein. In some embodiments, the modified corn has reduced or no wild type LMW-GS. In some embodiments, disclosed herein is a flour comprising any of the de-epitoped LMW-GS disclosed herein, wherein the flour is derived from the modified corn described herein.

In some embodiments, the modified wheat described herein can be manipulated such that expression of wild-type LMW-GS is down-regulated or eliminated. In some embodiments, the modified wheat may be used to generate other edible products such as beer.

In some embodiments, wheat is genetically modified to express any of the de-epitoped LMW-GS disclosed herein. In some embodiments, in wheat genetically modified to express a de-epitoped LMW-GS, the expression of the corresponding non-mutated LMW-GS polypeptide is down-regulated compared to a wild-type wheat. In certain embodiments, the genetically modified wheat comprises an RNA silencing agent directed towards the non-mutated polypeptide. In some embodiments, the genetically modified wheat is genetically modified by a DNA editing agent.

In some embodiments, a corn plant is genetically modified to express any of the de-epitoped LMW-GS disclosed herein. In some embodiments, in a corn plant genetically modified to express a de-epitoped LMW-GS, the expression of the corresponding non-mutated LMW-GS polypeptide is down-regulated compared to a wild-type corn plant

Food and beverage products comprising gluten may in certain embodiments, encompass breads, cakes, cookies, crackers, croutons, pastries, pasta, noodles, pizza, breakfast cereals, beer, ale, porter, stout, bulgur wheat, candies, communion wafers, French fries, gravies, imitation meat or seafood, malt, malt flavoring, matzo, hot dogs and processed lunchmeats, salad dressings, sauces including soy sauce, seasoned rice mixes, snack foods, self-basting poultry, soups, bouillon or soup mixes, vegetables in sauce, and the like. In some embodiments, a food product comprising gluten may be prepared from wheat, barley, rye, triticale (a cross between wheat and rye), or oats, or any combination thereof. Varieties of wheat that comprise gluten include durum, einkorn, emmer, kamut, and spelt.

In some embodiments, a food and or beverage product may comprise a processed food and or a processed beverage product.

Prescription and over-the-counter medications may use wheat gluten as a binding agent. In some embodiments, prescription and over-the counter medications may be gluten-free or have reduced gluten. In some embodiments, prescription and over-the counter medications comprise a modified LMW-GW polypeptide.

A skilled artisan would appreciate that the term “flour” may encompass a foodstuff which is a free-flowing powder, typically obtained by milling. Flour is most often used in bakery food products, such as breads, cakes, cookies, pastries etc., but also in other food products such as pasta, noodles, pizza, breakfast cereals and the like.

Examples of flour include bread flour, all-purpose flour, unbleached flour, self-rising flour, white flour, brown flour, and semolina flour. In some embodiments, there is provided a flour derived from a non-gluten plant, comprising at least one de-epitoped LMW-GS disclosed herein. In some embodiments, the non-gluten plant is transformed with a polynucleotide encoding a de-epitoped LMW-GS disclosed herein, and flour is generated therefrom (for example by grinding, mincing, milling etc.).

In some embodiments, flour is generated from a non-gluten plant (for example by grinding, mincing, milling etc.) and at least one de-epitoped LMW-GS disclosed herein is added.

A skilled artisan would appreciate that a non-gluten plant comprises a reduced quantity of gluten proteins. In some embodiments, a non-gluten plant comprises no or undetectable gluten proteins. In some embodiments, a non-gluten plant comprises between a 75%-100% reduction of gluten proteins. In some embodiments, a non-gluten plant comprises a 75%, 80%, 85%, 90%, 95%, or 99%, reduction of gluten proteins.

In some embodiments, disclosed herein is a flour generated from the wheat genetically modified to express at least one de-epitoped LMW-GS disclosed herein.

In some embodiments, disclosed herein is a dough generated from a wheat comprising a de-epitoped LMW-GS disclosed herein. In some embodiments, disclosed herein is a dough generated from a wheat comprising at least one de-epitoped LMW-GS disclosed herein. In certain embodiments, the dough described herein does not contain any wild type LMW-GS polypeptides. In certain embodiments, a dough is generated from any of the flours described herein.

The amount and variety of de-epitoped LMW-GS can be adjusted to change the quality of the flour or the dough generated therefrom. Thus, in some embodiments, use of any of a de-epitoped LMW-GS disclosed herein, or a combination thereof, improves a dough compared with a dough to which a de-epitoped LMW-GS has not been added. In some embodiments, the strengthening ability of the dough comprising the de-epitoped LMW-GS disclosed herein is not reduced by more than 50%, 60%, 70%, or 80% as compared to the dough comprising the wild-type LMW-GS.

A skilled artisan would appreciate that the term “dough” encompasses the commonly used meaning, namely, a composition comprising as minimal essential ingredients flour and a source of liquid, for example at least water that is subjected to kneading and shaping.

The dough of some embodiments may comprise additional components such as salt, plant starch, a flavoring agent, vegetable or vegetable part, oil, vitamins, and olives. The dough may further comprise a leavening agent, examples of which include unpasteurized beer, buttermilk, ginger beer, kefir, sourdough starter, yeast, whey protein concentrate, yogurt, biological leaveners, chemical leaveners, baking soda, baking powder, baker's ammonia, potassium bicarbonate and any combination thereof.

In some embodiments, a dough is combined with at least one additional food ingredient. In some embodiments, a dough is combined with at least one additional food ingredient comprising a flavoring agent, a vegetable, a vegetable part, a mix of vegetables, or a mix of vegetable parts, an oil, a plant starch, a vitamin, vitamins, or olives.

Processed products generated from the doughs comprising a de-epitoped LMW-GS described herein include, but are not limited to, pan bread, a pizza bread crust, a pasta, a tortilla, a Panini bread, a pretzel, a pie and a sandwich bread product.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of de-epitoped LMW-GS, methods producing same, and method of de-epitoping LMW-GS are presented in a range format, for example the number of antigenic units mutated, or the number of amino acids mutated within an antigenic unit. It should be understood that the description in range format is merely for convenience and brevity and should not be construed as an inflexible limitation on the scope of the invention. Accordingly, the description of a range should be considered to have specifically disclosed all the possible sub ranges as well as individual numerical values within that range. For example, description of a range such as from 1 to 6 should be considered to have specifically disclosed sub ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals there between.

In some embodiments, the term “about”, refers to a deviance of between 0.0001-5% from the indicated number or range of numbers. In some embodiment, the term “about”, refers to a deviance of between 1-10% from the indicated number or range of numbers. In some embodiments, the term “about”, refers to a deviance of up to 25% from the indicated number or range of numbers.

### EXAMPLES

Reference is now made to the following examples, which together with the above descriptions illustrate some embodiments of the de-epitoped low molecular weight (LMW) glutenin peptides in a non-limiting fashion.

Generally, the nomenclature used herein, and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Son s, Baltimore, Maryland (1989); Perbal, “A Practical Guide to Molecular Cloning”, John Wiley & Son s, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, CT (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Application s”, Academic Press, San Diego, CA (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Example 1

### Methods

### Computational Design of Peptides for Reduced HLA-DQ2.5 Binding

Close and more distantly related sequence homologs of low molecular weight glutenin subunit (uniprot accession D2DII2) were identified by searches performed at the protein level, by either a blast search (Altschul S. F. et al., NAR 25.17 (1997)) or by HHblits, which employs a Hidden Markov Model (HMM)-based iterative sequence search (Remmert M. et al., Nature methods 9.2 (2012)). Sequences with an expected value greater than 10⁻¹⁰ were discarded. Next, sequences were aligned using Clustal Omega (Sievers, F. et al., Molecular systems biology 7.1 (2011)), followed by identification and extraction of all sequence segments aligned to the epitopes of interest.

The design of peptides predicted to bind HLA-DQ2.5 with reduced affinity was performed by in-silico structural modeling and energy calculations. The solved crystal structure of HLA-DQ2.5 bound to alpha gliadin epitopes DQ2.5-glia-ala (PDB accession 4ozi) was used as a starting structure. The structure of epitopes glut-L1 (amino acid sequence QPPFSEQEQPV, SEQ ID NO:188) and glut-L2 (amino acid sequence QPPFSEQQESP, SEQ ID NO:189) were modeled by simple sidechain mutating of the alpha-gliadin peptide structure, using the “mutate residues” module. Structural refinement was performed on the starting structures using the Protein Preparation Wizard. Next, using the Residue Scanning tool, monte-carlo sampling was performed in search of peptide sequences where conserved mutations were combined and the predicted change in binding energy upon mutation, ΔΔG, was calculated. Candidate peptide sequences were selected for experimental validation based on the predicted change in binding energy (where an increase of at least 10 relative energy units was required) and manual inspection of the generated interaction models.

### In-Vitro Testing of MHC Class H Binding by Modified Gluten Peptides

In-vitro testing of modified gluten peptides was performed by a Major histocompatibility complex (MHC class II) binding assay (Sidney J. et. al. Current protocols in immunology 100.1 (2013)). Briefly, competition assays using different concentrations of WT and modified low molecular weight glutenin subunit (LMW-GS) peptides were conducted by diluting the peptides in NP40 buffer, and incubation for 2-4 days with purified MHC and a radiolabeled known MHC binding peptide (peptide probe). MHC class II molecules were purified by affinity chromatography, and peptides were radiolabeled using the chloramine T method. After the incubation period, the bound and unbound radiolabeled species were separated, and their relative amounts were determined by either size-exclusion gel-filtration chromatography or monoclonal antibody capture of MHC. The percentage of bound radioactivity was then determined. For each modified peptide, IC₅₀ of WT and modified peptide were calculated. The known gluten peptide epitopes were analyzed for MHC binding as a positive control, both in the non-deamidated and in the deamidated form. Any IC₅₀ value greater than 4-fold relative to the IC₅₀ of the WT peptide signifies that the binding of the engineered peptide chain is compromised.

Purification of Gluten Proteins Expressed in E. coli

A high throughput method for screening a large number of proteins was applied. Briefly, plasmid harboring WT or mutant LMW-GS proteins was generated. Transcription was under the control of a combination of a lac-operator and a T7-promoter, which allows for effective repression or induction with IPTG. E. coli cells were grown at 37° C. and transferred to minimal medium. Expression of LMW-GS proteins were induced by adding IPTG. Following induction, cells were lysed, and total cell lysate was spotted on a nitrocellulose membrane. The membrane was then blocked with skim milk. His tagged proteins were probed with an anti His antibody. After identifying LMW genes with high expression, a new construct was prepared, in which the HIs-tag was removed.

### Generation of Gluten-Specific T Cell Lines

Fractionation of Gluten Proteins from Wheat Flour

Low molecular weight glutenins (LMW) were prepared from wheat flour as described by Schalk et. al. (PLOS ONE 12, 2017) and Melas et. al. (Cereal chemistry 71, 1994). Briefly, flour was defatted using n-pentane/ethanol solution. Defatted flour was extracted with salt solution. The sediment was extracted with ethanol/water and the resulting supernatant was concentrated and dialyzed against 0.01M acetic acid and lyophilized (proalmin fraction). The remaining sediment was extracted with 2-propanol/water (50/50, v/v)/0.1 mol/l Tris-HCl, pH 7.5, containing 2 mol/L (w/v) urea and 0.06 mol/L (w/v) dithiothreitol (DTT)) for 30 min at 60° C. Following centrifugation, the supernatant was collected, and acetone was added to a final concentration of 40% (V/V). The mixture was allowed to sit at room temperature for 10 minutes and then centrifuged to precipitate the High molecular weight glutenin (HMW) fraction. Finally, the acetone concentration in the supernatant was adjusted to 80% (V/V), and the supernatant was allowed to sit at room temperature for 10 minutes. Then, the mixture was centrifuged to collect the LMW fraction.

Purification of Recombinant LMW from E. coli for T Cell Assay

Bacterial pellet expressing the LMW protein was resuspended in buffer A (50 mM Tris pH 9, 50 mM NaCl) and cells were lysed using sonication. Cell lysate was centrifuged, and pellet was washed with buffer A. Pellet was solubilized with buffer E.B (8M Urea, 10 mM DTT, 100 mM acetic acid) and incubated at 65° C. for 1 hour with vortex every 10 mins. The sample was centrifuged and supernatant was transferred into a new bottle. DDW was added to supernatant at 1:1 ratio and incubated for 15 minutes at room temperature. The sample was centrifuged and supernatant was transferred to a new bottle. Acetone was added up to 80% and incubated for 18 hours at 4° C. Approximately 80% of the supernatant was discarded and the remaining volume was centrifuged to form a pellet containing LMW protein.

### Pepsin Chymotrypsin Digestion of LMW for T Cell Assay

Two hundred mg of protein were first incubated in 5% formic acid with 4 mg pepsin for 4 hours in 37° C. Then the sample was evaporated using speedVac. On the following day, the protein was incubated with 4 mg chymotrypsin in 20 mM ammonium-bicarbonate for 4 hours in 37° C. followed by speedVac evaporation. The concentration of the digest was then measured, and the digest was deamidated using transglutaminase, 2U per 7 mg digest for 6 hours in 37° C. The samples were then cleaned using a C18 column, dried and resuspended in media at a concentration of 2 mg/ml.

### Biopsy Processing for T Cell Lines Generation

Gluten-reactive T cell lines (TCLs) were generated based on a previously described method, with modifications (Gianfrani C. et al., Gastroenterology 133.3 (2007)). Briefly, mucosal explants were digested with collagenase A and cells were seeded at 2-3×10⁵ cells/ml in complete medium X-Vivo15 (Lonza) supplemented with 5% AB-pooled human serum (Biotag) and antibiotics. Cells were stimulated with 1.5×10⁶ irradiated PBMC and TG2 (Sigma-Aldrich)-treated (deamidated) pepsin chymotrypsin (PCT-) digested LMW (50 μg/ml). IL-15 and IL-2 (Peprotech) were added after 24 h at 10 ng/ml and 20 units/ml respectively. Cytokines were supplemented every 3-4 days and cells were split according to need. The cells were restimulated approximately 2 weeks after the first stimulation.

### T Cell Assay

TCLs were assayed for responses to deamidated PCT-LMW proteins and LMW peptides by the detection of IFN-γ by enzyme-linked immunosorbent assay (ELISA) as previously described (Gianfrani C. et al., J. Immunol. 177.6 (2006)). HLA-matched B-LCLs (Sigma-Aldrich) or orthologous PBMCs were used as antigen presenting cells (APCs). PCT-LMW proteins (100 g/ml) or LMW peptides (10 μM) (A&A labs) were added to APCs (1×10⁵) concomitantly with responder T cells (4×10⁴), the cells were seeded in 200 μl X-vivo15 medium in round-bottom 96 well plate (Corning) and incubated for 48 h. Each peptide/protein was tested in 4 replicates. DMSO serves as negative control for peptides testing and blank medium serves as negative control for protein testing. For ELISA experiments, Nunc MaxiSorp plates (Thermo Fisher) were coated with 1 μg/ml α-IFNγ antibody (Mabtech), blocked and incubated overnight with 50 μl of the sups taken from the TCLs' plates. Recombinant IFNγ (Bactlab) was used for standard curve generation. The plate was incubated with biotin-α-IFNγ antibody (1 μg/ml) (Mabtech), streptavidin-HRP (Bactlab)(1:5000) and TMB (Thermo Fisher). The reaction was stopped, and the plate was read on the ELISA plate reader at 450 nM. The results were analyzed using Graphpad Prism and IFNγ levels were determined. The results were normalized to the control. Results were considered positive (activating T cells) if IFNγ levels were >2-fold in peptide/protein samples compared to control or if IFNγ levels were significantly higher than the control (one-sided student t-test).

### Protein Purification for Biophysical Qualities Assessment

Bacterial pellet expressing the LMW protein was resuspended in lysis buffer (50 mM Tris pH 9, 50 mM NaCl) and cells were lysed using pressure homogenizer. Cell lysate was centrifuged, pellet was collected and washed with wash buffer (50 mM Tris pH=8, 2.5 mM EDTA and 1% Tween 20). The pellet was washed with 0.1 mM acetic acid for 1 hour and the pellet was collected. Finally, the protein pellet was freeze dried and grinded to powder.

### Assessment of Biophysical Qualities of De-Epitoped Gluten Gene Sets

Each recombinant protein (WT or de-epitoped variants) was tested separately and in combination to determine the contribution of individual proteins and specific combinations to different biophysical characteristics. Recombinant HMW-GS and LMW-GS were purified from expression hosts. Macro polymer formation efficiency was analyzed by SDS-PAGE and size exclusion chromatography as described by others (Veraverbeke et al., Cereal Chemistry. 77.5 (2000a, 2000b)). Different combinations and quantities of different recombinant gluten proteins were tested to achieve optimal dough and bread properties. Dough was produced by mixing purified recombinant gluten proteins with starch and biophysical properties were assessed for example with farinograph and alveograph (Testing parameters: mixing properties dough development time and peak consistency values). Baked breads were tested for volume, crumb color and texture attributes, resilience, and adhesiveness (Patra cu et al., Food Sci Technol Int. 23.2 (2017); Uthayakumaran et al., Cereal Chemistry 77.6 (2000)).

### Results

Tables I and II list the measured IC₅₀ values from competition assays between the specified peptides and an MHC class II bound probe peptide.

TABLE I

| | HLA- | HLA- | HLA- | HLA- | |
|---|---|---|---|---|---|
| | DQ2.5 | DQ8 | DQ2.2 | DQ7.5 | |
| Sequences | IC₅₀ (nM) | IC₅₀ (nM) | IC₅₀ (nM) | IC₅₀ (nM) | Comments |
| QPPFSQQQQSPESQQ | 31558 | 12774 | Not tested | Not tested | Glut-L2 |
| (SEQ ID NO: 68) | | | | | epitope |
| QPPFSEQQESPFSQQ | 158 | 1142 | Not tested | Not tested | Deamidated |
| (SEQ ID NO: 69) | | | | | Glut-L2 |
| QPPSSEQQHSPPSQQ | >40000 | 31882 | 39773 | >40000 | |
| (SEQ ID NO: 81) | | | | | |
| QQPFPEQQRSQFSQQ | >40000 | 35785 | >40000 | >40000 | |
| (SEQ ID NO: 85) | | | | | |
| QSPFSRQQKSPFSQQ | >40000 | >40000 | >40000 | >40000 | |
| (SEQ ID NO: 80) | | | | | |
| QPSFSRQQKQPQSQQ | >40000 | >40000 | >40000 | >40000 | |
| (SEQ ID NO: 86) | | | | | |
| QPPDSHQQHSPGSQQ | >40000 | >40000 | >40000 | >40000 | |
| (SEQ ID NO: 84) | | | | | |
| QPPFSEQQEPSSQQP | >40000 | >40000 | 10629 | Not tested | |
| (SEQ ID NO: 87) | | | | | |
| QPPFSHQRPPFSQQQ | >40000 | >40000 | Not tested | Not tested | |
| (SEQ ID NO: 82) | | | | | |
| QPFFSHQQRSGFSQQ | >40000 | 39185 | Not tested | Not tested | |
| (SEQ ID NO: 83) | | | | | |
| QSPFPEQQEQHQQLQ | >40000 | 37899 | >40000 | >40000 | |
| (SEQ ID NO: 185) | | | | | |
| GPPFSKQTEPVISQQ | >40000 | 38179 | >40000 | >40000 | |
| (SEQ ID NO: 186) | | | | | |

TABLE II

| | HLA- | HLA- | HLA- | HLA- | |
|---|---|---|---|---|---|
| | DQ2.5 | DQ8 | DQ2.2 | DQ7.5 | |
| Sequences | IC₅₀ (nM) | IC₅₀ (nM) | IC₅₀ (nM) | IC₅₀ (nM) | Comments |
| QQPPFSQQQQPVLPQ | 39972 | 36771 | Not tested | Not tested | Glut-L1 |
| (SEQ ID NO: 66) | | | | | epitope |
| QQPPFSEQEQPVLPQ | 3048 | 1106 | Not tested | Not tested | Deamidated |
| (SEQ ID NO: 67) | | | | | Glut-L1 |
| QQPPFPRQHQQFPQQ | >40000* | >40000 | >40000 | >40000 | |
| (SEQ ID NO: 74) | | | | | |
| QQSPFSRQEKSPFSQ | >40000 | >40000 | >40000 | >40000 | |
| (SEQ ID NO: 78) | | | | | |
| QQPPFSKQEQQQPIL | >40000 | 29794 | >40000 | >40000 | |
| (SEQ ID NO: 71) | | | | | |
| QGPPFSKQTQPVIPQ | >40000 | >40000 | >40000 | >40000 | |
| (SEQ ID NO: 72) | | | | | |
| QQPAFSRQHQPSLPQ | 30387 | >40000 | >40000 | >40000 | |
| (SEQ ID NO: 73) | | | | | |
| QQPPSSEQEHSPPSQ | >40000 | >40000 | >40000 | >40000 | |
| (SEQ ID NO: 79) | | | | | |
| QQPPFSHQRPPFSQQ | >40000 | 39052 | >40000 | >40000 | |
| (SEQ ID NO: 70) | | | | | |
| QQPFPQEQRSQFSQQ | >40000 | >40000 | Not tested | Not tested | |
| (SEQ ID NO: 75) | | | | | |
| QQPPFWQHQPPFSQQ | 37352 | Not | Not tested | Not tested | |
| (SEQ ID NO: 76) | | tested | | | |
| QQPPFSEQEQPSSQQ | 32348 | 31499 | Not tested | Not tested | |
| (SEQ ID NO: 77) | | | | | |
| QQSPFPEQEQQHQQL | 32404 | 39928 | >40000 | >40000 | |
| (SEQ ID NO: 183) | | | | | |
| QQPPDSHQEHSPGPQ | >40000 | >40000 | >40000 | >40000 | |
| (SEQ ID NO: 187) | | | | | |

The results presented in Tables I and II demonstrate that unlike the WT peptides, the engineered peptides are unable to efficiently compete with the radiolabeled probe peptide for binding to the tested MHC class II molecules. In Celiac disease, a necessary step in the process of eliciting an immune response is the binding of digested gluten peptides to at least one of the tested MHC class II molecules. The presented results suggest that a replacement of antigenic units within LMW-GS proteins with any of the designed peptides would result in a reduction in protein immunogenicity.

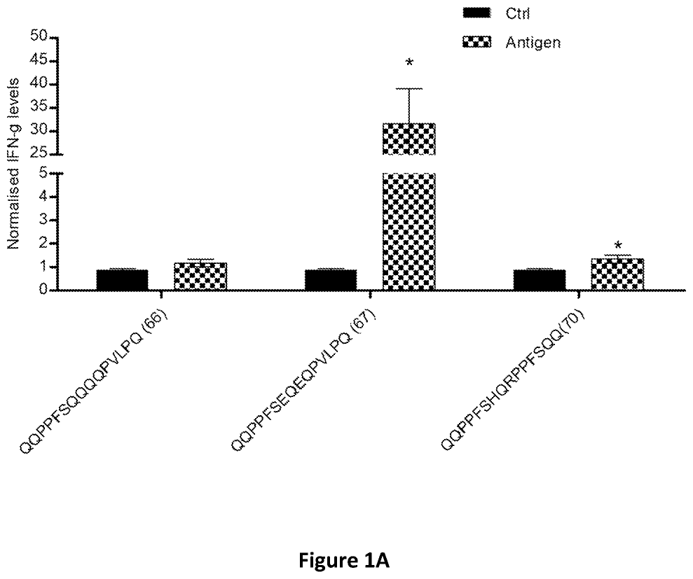

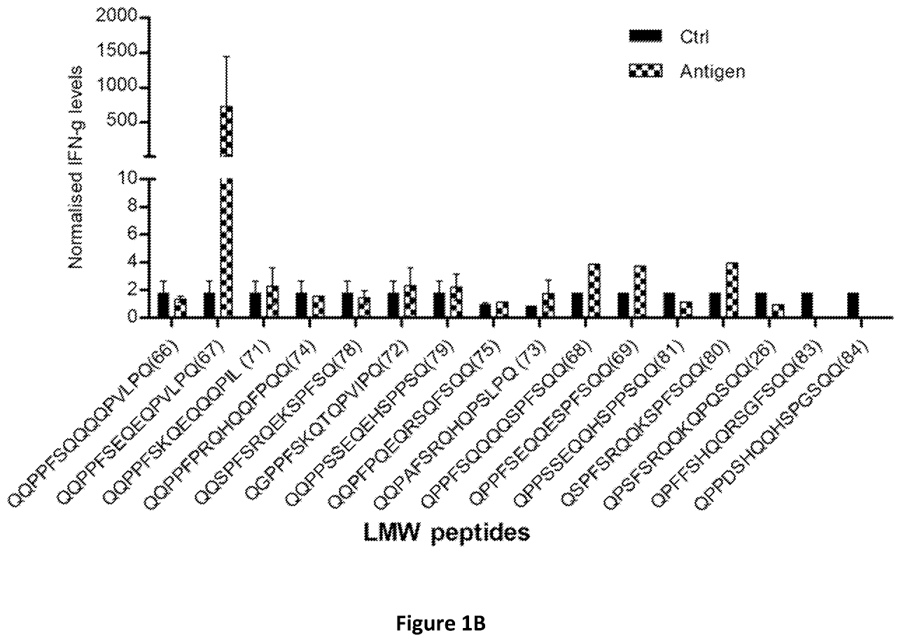

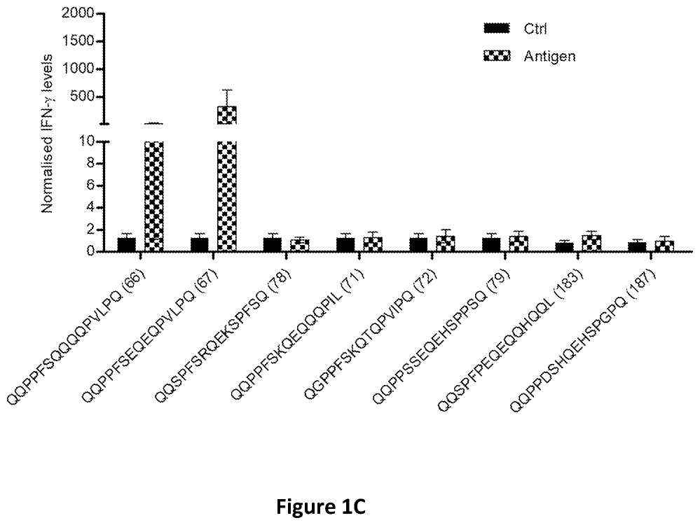

FIGS. 1A-IC show that modifications to LMW-GS immunogenic peptide led to abolishment of T cell activation. Mean response to tested LMW-GS WT and modified peptides of TCLs from a patient biopsy were assayed by an ELISA assay, detecting levels of IFN-γ. The TCL response to LMW-GS was considered positive when normalized IFN-γ production was significantly higher for a tested peptide compared to a DMSO control. The results presented in the FIGURES demonstrate that unlike the WT peptides, the engineered peptides do not activate T cells to a level that is statistically different than the baseline activation (the DMSO control). The presented results suggest that a replacement of antigenic units within LMW-GS proteins with any of the designed peptides would result in a significant reduction in protein immunogenicity.

1. A de-epitoped low molecular weight glutenin subunits (LMW-GS) derived from a wild-type LMW-GS, said de-epitoped LMW-GS comprises one or more modified epitopes, wherein the modified epitopes comprise the amino acid sequence of X1, X2, X3, X4, Q, X6, X7, X8, X9, X10, wherein
X1 is P, A, S, or null,
X2 is F, S, or D,
X3 is S or P,
X4 is Q, R, K, H, or E,
X6 is Q, R, T, H, or E,
X7 is Q, R, K, H, P, or E,
X8 is P, S, or Q,
X9 is P, V, Q, H, F, S, or G, and
X10 is F, P, Q, S, G, I, or null.

2. The de-epitoped LMW-GS of claim 1, wherein the LMW-GS comprises at least two modified epitopes.

3. The de-epitoped LMW-GS of claim 1, wherein the modified epitope comprises the amino acid sequence of X1, X2, X3, X4, Q, X6, X7, X8, X9, wherein
X1 is P, A, or S,
X2 is F, S, or D,
X3 is S or P,
X4 is Q, R, K, H, or E,
X6 is Q, R, T, H, or E,
X7 is Q, R, K, H, P, or E,
X8 is P, S, or Q, and
X9 is P, V, Q, H, F, S, or G.

4. The de-epitoped LMW-GS of claim 1, wherein the modified epitope comprises the amino acid sequence of X1, X2, X3, Q, X5, X6, X7, X8, X9, wherein
X1 is F, S, or D,
X2 is S or P,
X3 is Q, R, K, H, or E,
X5 is Q, R, T, H, or E,
X6 is Q, R, K, H, P, or E,
X7 is P, S, or Q,
X8 is P, V, Q, H, F, S, or G, and
X9 is F, P, Q, S, G, or I.

5. The de-epitoped LMW-GS of claim 1, wherein the modified epitope comprises the amino acid sequence of one of SEQ ID NOs:11, 12, 14, 15, 17, 18, 19, 20, 71, 72, 78, 79, 80, 81, 83, 84, 91, 92, 94, 95, 98, 101, 102, 104, 105, 108, 110, 111, 119-160, 175, 176, 177, 178, 179, 180, 181, 182, 183, 184,185, 186 and 187.

6. (canceled)

7. (canceled)

8. The de-epitoped LMW-GS of claim 1, wherein the wild-type LMW-GS comprises the amino acid sequence of one of SEQ ID NOs:88, 89, 90, 112-118, and 161-174, or the amino acid sequence having at least 60% identify with one of SEQ ID NOs:88, 89, 90, 112-118, and 161-174.

9. An isolated polynucleotide encoding the de-epitoped LMW-GS of claim 1.

10. An expression vector comprising the isolated polynucleotide of claim 9, operatively linked to a transcriptional regulatory sequence to allow expression of said de-epitoped LMW-GS in a cell or in a cell-free in vitro system.

11. (canceled)

12. The expression vector of claim 10, wherein said transcriptional regulatory sequence comprises a plant promoter.

13. (canceled)

14. A cell comprising at least one de-epitoped LMW-GS of claim 1.

15. (canceled)

16. A method of producing the de-epitoped LMW-GS of claim 1, said method comprising expressing an expression vector comprising an isolated polynucleotide encoding said de-epitoped LMW-GS in a cell, and collecting expressed LMW-GS from said cell.

17. (canceled)

18. A modified wheat comprising the de-epitoped LMW-GS of claim 1.

19. A flour comprising the de-epitoped LMW-GS of claim 1.

20. A dough comprising the flour of claim 19.

21. A food or beverage product comprising the de-epitoped LMW-GS of claim 1.

22. (canceled)

23. A method of de-epitoping LMW-GS, comprising changing one or more amino acid residues of at least one epitope of a wild type LMW-GS, said epitope comprises the amino acid sequence of X1, X2, X3, X4, Q, X6, X7, X8, X9, wherein
X1 is changed to P, A, or S,
X2 is changed to F, S, or D,
X3 is changed to S or P,
X4 is changed to Q, R, K, H, or E,
X6 is changed to Q, R, T, H, or E,
X7 is changed to Q, R, K, H, P, or E,
X8 is changed to P, S, or Q, and
X9 is changed to P, V, Q, H, F, S, or G,
thereby generating a de-epitoped LMW-GS comprising one or more modified epitopes.

24. The method of claim 23, wherein said epitope of a wild type LMW-GS comprises the amino acid sequence of one of SEQ ID NOs:21-48.

25. A method of de-epitoping LMW-GS, comprising changing one or more amino acid residues of at least one epitope of a wild type LMW-GS, said epitope comprises the amino acid sequence of X1, X2, X3, Q, X5, X6, X7, X8, X9, wherein
X1 is changed to F, S, or D,
X2 is changed to S or P,
X3 is changed to Q, R, K, H, or E,
X5 is changed to Q, R, T, H, or E,
X6 is changed to Q, R, K, H, P, or E,
X7 is changed to P, S, or Q,
X8 is changed to P, V, Q, H, F, S, or G, and
X9 is changed to F, P, Q, S, G, or I,
thereby generating a de-epitoped LMW-GS comprising one or more modified epitopes.

26. The method of claim 25, wherein said epitope of a wild type LMW-GS comprises the amino acid sequence of one of SEQ ID NOs:50-64.

27. The method of claim 23, wherein the method further comprises one or more of
(i) analyzing binding affinity of said modified epitopes of the de-epitoped LMW-GS to one or more of MHC Class II molecules to identify a de-epitoped LMW-GS with reduced binding affinity to the MHC Class II molecules; or
(ii) analyzing immunogenicity of said de-epitoped LMW-GS to identify a de-epitoped LMW-GS with reduced immunogenicity
wherein the MHC Class II molecule is HLA-DQ2.5, HLA-DQ8, HLA-DQ2.2, or HLA-DQ7.5.

28. (canceled)
